(19) United States
(12) Patent Application Publication
(10) Pub. No.: US 2024/0269144 A1
(43) Pub. Date: Aug. 15, 2024

(54) METHODS AND KITS FOR THE DETECTION OF MALIGNANCIES

(71) Applicants: H. LEE MOFFITT CANCER CENTER AND RESEARCH INSTITUTE, INC., Tampa, FL (US); BOARD OF REGENTS, THE UNIVERSITY OF TEXAS SYSTEM, Austin, TX (US)

(72) Inventors: Pei-Ling CHEN, Tampa, FL (US); Xiaofei SONG, Tampa, FL (US)

(21) Appl. No.: 18/566,277

(22) PCT Filed: Jun. 2, 2022
(86) PCT No.: PCT/US2022/031991

§ 371 Date: Dec. 1, 2023

Related U.S. Application Data
(60) Provisional application No. 63/195,985, filed on Jun. 2, 2021.
(60) Provisional application No. 63/208,844, filed on Jun. 9, 2021.

(51) Int. Cl. A61K 31/519; A61K 31/415; A61K 31/454; A61K 31/5415; A61P 35/00; C12Q 1/6869; C12Q 1/6886

(52) U.S. Cl. CPC A61K 31/519; A61K 31/415; A61K 31/454; A61K 31/5415; A61P 35/00; C12Q 1/6869; C12Q 1/6886; C12Q 2600/158

(57) ABSTRACT

Disclosed novel expression and mutation patterns that correlate with the presence of a malignancy in B and T cell cancers. Accordingly, disclosed herein are kits and methods for the diagnosis of cancer malignancies.

This application claims the benefit of U.S. Provisional Application No. 63/208,844, filed on Jun. 9, 2021, and U.S. Provisional Application No. 63/195,985, filed on Jun. 2, 2021, applications which are incorporated herein by reference in their entirety.

This invention was made with government support under Grant No. CA240434 and CA076292 awarded by the National Institutes of Health. The government has certain rights in the invention.

### I. BACKGROUND

Cutaneous T-cell lymphomas are part of a group of non-Hodgkin lymphomas characterized by skin-homing malignant T-cells. The most common form of CTCL, mycosis fungoides (MF, 50-60% of CTCL), typically behaves as a CD4+ indolent lymphoma at presentation and is characterized by cutaneous manifestations of patch/plaque lesions, while Sezary syndrome (SS, leukemic form, 2-3% of CTCL) typically manifests as generalized erythroderma with circulating malignant T-cells in the peripheral blood. Transformation of CTCL occurs when there is a change in histopathologic appearance from neoplastic small-medium lymphocytes to large, blast-like T-cells. The primary site of detectable transformation is usually in the skin (>92%). Similar to Richter's transformation in chronic lymphocytic leukemia/small cell lymphoma, large cell transformation (LCT) in CTCL heralds transition to aggressive clinical behavior and a rapid decline in survival despite multi-modality treatment regimens (<2 years in ˜50-60% of patients).

Major advances have been made in the treatment of advanced stage disease with the FDA approval of mogamulizumab (anti-CCR4) as a breakthrough therapy for CTCL in 2018. Nonetheless, majority of the patients did not respond to mogamulizumab monotherapy (MAVORIC trial³; ORR 25.3% at ≥4 months, median PFS 7.8 months), and responses were compartmentalized, with responses in blood and skin seen in 49.2% and 27.4% of patients⁴, respectively. The MAVORIC trial thus highlights a critical need to identify therapeutic targets for advanced stage disease in the skin compartment.

### II. SUMMARY

Disclosed are methods and compositions related to the detection of malignancies in T cell and B cell cancers.

In one aspect, disclosed herein are methods of detecting malignant cells in a tumor microenvironment comprising obtaining a tissue sample (such as, for example, tissue samples comprising T cells and B cells including, but not limited to B cell and T cell tumor samples, as well as tissue, tissue samples comprising dendritic cells monocytes, and endothelial cells), performing w % hole exome sequencing (WES), parallel single-cell RNAseq, single cell TCRseq and/or copy number variation (CNV) interference assay on the sample; wherein the pattern of increase or decrease in gene expression and/or the presence of mutations relative to a control indicates that the subject does or does not have a malignancy.

Also disclosed herein are methods of detecting malignant cells in a tumor microenvironment of any preceding aspect, wherein a malignancy is indicated by mutations in genes related to Oxidative phosphorylation, MYC targets V1, MYC targets V2, epithelial mesenchymal transition, E2F targets, xenobiotic metabolism, coagulation, WNTb catenin signaling, cholesterol homeostasis, spermatogenesis, estrogen response late, DNA repair.

In one aspect, disclosed herein are methods of detecting malignant cells in a tumor microenvironment of any preceding aspect, wherein the cancer is Cutaneous T-cell lymphoma (CTCL) and the presence of a malignancy is indicated by an increase in 5 or more of the genes selected from the list consisting of CD9, CD74, COPA, APP, RAB25, LSR, CLDN7, EPCAM, TWIST1, LGALS3, S100A6, SLC25A5, PIM3, I122, PTHLH, CCR7, AHR, CORO1 B, ROMO1, MIF, NME2, NDUFB2, SRM, L AIR2, HTRA1, PAGE.5, IGFBP2, AREG, XCL2, and XCL1; and a decrease in the expression of 5 or more of the genes consisting of RGCC, ANXA1, FOS, ZFP36, HLA-DPB1, HLA-DPAL, HLA-DRB1, SAT1, FTH11, HSPA1B, DNAJB1, GLIPR1, IL10RA, SELL, TXNIP, ARID5B, RHOH, BTG1, TPT1, HLA-B, HLA-A, HLA-F, TNFRSF4, IL2RA, and GBP5.

Also disclosed herein are disclosed herein are methods of detecting malignant cells in a tumor microenvironment of any preceding aspect, wherein the cancer malignancy is mycosis fungoides (MF)/cutaneous T-cell lymphoma, wherein at least on assay comprises copy number variation (CNV), and the presence of the malignancy is indicated by an increase in the expression of four or more of the genes selected from the group consisting of SYCP1, TM4SF1, AHR, NDUFB2, TWIST1, LGALS3, RBBP6, and NDUFA7 and a decrease of six or more of the genes selected from the group consisting of PRAMEF, PCDHGB, PMS2P1, RECQL4, IFNA, CDKN2, NOTCH1, HRAS, POLE, AKT1, DEFB, and SOCS7.

In one aspect disclosed herein are methods of detecting malignant cells in a tumor microenvironment of any preceding aspect, wherein the cancer malignancy is mycosis fungoides (MF) and the presence of the malignancy is indicated by a mutation in three of more of the genes selected from the group consisting of FSIP2, TEKT4, PCDH15, TP53, STAT3, JAK3, KMT2C, CREBBP, ARID1A, TRRAP, KMT2D, PLCG1, and CDKN2A.

Also disclosed herein are methods of detecting malignant cells in a tumor microenvironment of any preceding aspect, wherein the cancer malignancy is leukemic CTLC; wherein at least on assay comprises copy number variation (CNV), and the presence of malignancy is indicated by an increase in the expression of CARD11 and/or MUC16 and a decrease in the expression of 5 or more of the genes selected from the group consisting of ARID1A, TNFAIP3, CDKN2A, DAPL1, WT1, ATM, CDKN1B, SOCS2, RB1, and STK11.

In one aspect, disclosed herein are disclosed herein are methods of detecting malignant cells in a tumor microenvironment of any preceding aspect, wherein when a malignant cell is detected in the tumor microenvironment, the method further comprises administering to the subject one or more anti-cancer agents (such as, for example, an OXPHOS (including but not limited to IACS-010759), Myc (including but not limited to MYCi975), MIF (including but not limited to BTZO-1), and/or CDK4/6 inhibitor (including, but not limited to Palbciclib).

Also disclosed herein are methods of diagnosing or detecting a cancer or cancer type in a subject comprising obtaining a tissue sample (such as, for example, tissue samples comprising T cells and B cells including, but not limited to B cell and T cell tumor samples, as well as tissue, tissue samples comprising dendritic cells monocytes, and endothelial cells), performing whole exome sequencing (WES), parallel single-cell RNAseq, single cell TCRseq and/or copy number variation (CNV) interference assay on the sample; wherein the pattern of gene expression indicates the type of cancer the subject has.

In one aspect, disclosed herein are methods of diagnosing or detecting a cancer or cancer type of any preceding aspect, wherein the presence of a Cutaneous T-cell lymphoma (CTCL) is indicated by an increase in 5 or more of the genes selected from the list consisting of CD9, RAB25, LSR, CLDN7, EPCAM, TWIST1, LGALS3, S100A6, SLC25A5, PIM3, IL22, PTHLH, CCR7, AHR, CORO1B, ROMO1, MIF, NME2, NDUFB2, SRM, LAIR2, HTRA1, PAGE5, IGFBP2, AREG, XCL2, and XCL1; and a decrease in the expression of 5 or more of the genes consisting of RGCC, ANXA1, FOS, ZFP36, HLA-DPB1, HLA-DPA1, HLA-DRB1, SAT1, FTH1, HSPA1B, DNAJB1, GLIPR1, IL10RA, SELL, TXNIP, ARID5B, RHOH, BTG1, TPT1, HLA-B, HLA-A, HLA-F, TNFRSF4, IL2RA, and GBP5.

Also disclosed herein are methods of diagnosing or detecting a cancer or cancer type of any preceding aspect, wherein at least on assay comprises copy number variation (CNV), and the presence of a leukemic CTLC is indicated by an increase in the expression of CARD11 and/or MUC16 and a decrease in the expression of 5 or more of the genes selected from the group consisting of ARID1A, TNFAIP3, CDKN2A, DAPL1, WT1, ATM, CDKN1B, SOCS2, RB1, and STK11.

In one aspect, disclosed herein are methods of diagnosing or detecting a cancer or cancer type of any preceding aspect, wherein at least on assay comprises copy number variation (CNV), and the presence of a mycosis fungoides (MF) is indicated by an increase in the expression of four or more of the genes selected from the group consisting of SYCP1, TM4SF1, AHR, NDUFB2, TWIST1, LGALS3, RBBP6, and NDUFA7 and a decrease of six or more of the genes selected from the group consisting of PRAMEF, PCDHGB, PMS2P1, RECQL4, IFNA, CDKN2, NOTCH1, HRAS, POLE, AKT1, DEFB, and SOCS7.

Also disclosed herein are methods of diagnosing or detecting a cancer or cancer type of any preceding aspect, wherein the presence of a mycosis fungoides (MF) is indicated by a mutation in three of more of the genes selected from the group consisting of FSIP2, TEKT4, PCDH15, TP53, STAT3, JAK3, KMT2C, CREBBP, ARID1A, TRRAP, KMT2D, PLCG1, and CDKN2A.

In one aspect, disclosed herein are disclosed herein are methods of diagnosing or detecting a cancer or cancer type of any preceding aspect, wherein when a diagnosis of cancer is made or a cancer is detected or a cancer type is determined (such as, for example, the detection and or diagnosis of Cutaneous T-cell lymphoma (CTCL), melanoma, and/or breast cancer (including, but not limited to TNBC), the method further comprises treating the subject comprising administering to the subject one or more anti-cancer agents (such as, for example, an OXPHOS, Myc, MIF, and/or CDK4/6 inhibitor (including, but not limited to Palbciclib).

Also disclosed herein are methods of treating, inhibiting, reducing, decreasing, ameliorating, and/or preventing a cancer and/or metastasis in a subject comprising obtaining a tissue sample, performing whole exome sequencing (WES), parallel single-cell RNAseq, single cell TCRseq and/or copy number variation (CNV) interference assay on the sample; wherein the pattern of increase or decrease in gene expression and/or the presence of mutations relative to a control indicates that the subject has a cancer, malignancy, and/or the cancer type; and wherein a cancer, malignancy, and/or the type of cancer in the subject is detected, administering to the subject one or more anti-cancer agents (such as, for example, , an OXPHOS (including but not limited to IACS-010759), Myc (including but not limited to MYCi975), MIF (including but not limited to BTZO-1), and/or CDK4/6 inhibitor (including, but not limited to Palbciclib).

Also disclosed herein are kits for diagnosing a cancer in a subject or detecting a malignancy in a subject comprising primers and/or probes for the detection of mutations or the expression of five or more genes selected from the group consisting of CD9, RAB25, LSR, CLDN7, EPCAM, TWTST1, LGALS3, S100A6, SLC25A5, PIM3, 1L22, PTHLH, CCR7. AHR, CORO1B, ROMO1, MIF, NME2, NDUFB2, SRM, LAIR2, HTRA1 PAGE5, IGFBP2, AREG, XCL2, XCL1, RGCC, ANXA1, FOS, ZFP36, HILA-DPB1, HLA-DPA1, HILA-DRB1, SAT1, FTH1, HSPA1B, DNAJB1, GLTPR1, IL10RA, SELL, TXNIP, ARID5B, RHOH, BTG1, TPT1, HLA-B, HLA-A, HLA-F, 1 TRSF4, IL2RA, GBP5, FSIP2, TEKT4, PCDH15, TP53, STAT3, JAK3, KMT2C, CREBBP, ARID1A, TRRAP, KMT2D, PLCG1, CDKN2A, SYCP1, TM4SF1, AHR, NDUFB2, TWIST1, LGALS3, RBBP6, NDUFA7, PRAMEF, PCDHGB, PMS2P1, RECQL4, IFNA, CDKN2, NOTCH1, HRAS, POLE, AKT1, DEFB, SOCS7, CARD11, MUC16, ARID1A, TNFAIP3, CDKN2A, DAPL1, WT1, ATM, CDKN1B, SOCS2, RB1, and STK11.

In one aspect, disclosed herein are methods of detecting a malignancy or assessing the severity of a cancer comprising obtaining a tissue sample (such as, for example, tissue samples comprising T cells and B cells including, but not limited to B cell and T cell tumor samples, as well as tissue, tissue samples comprising dendritic cells monocytes, and endothelial cells), performing whole exome sequencing (WES), parallel single-cell RNAseq, single cell TCRseq and/or copy number variation (CNV) interference assay on the sample; wherein a poor prognosis (i.e., a more sever cancer or more malignant cancer) is indicated by the co-localization of MIF and CD74, CD74 and COPA, CD74 and APP, HLA-B and KIR3DL2, and/or HLA-C and FAM3C. In one aspect, the method can further comprise treating the cancer with a MIF inhibitor.

Also disclosed herein are methods of treating, inhibiting, decreasing, reducing, ameliorating a cancer and/or malignancy in a subject (such as, for example, a T-cell lympohoma) comprising administering to the subject a MIF inhibitor. In one aspect, disclosed herein are methods of treating, inhibiting, decreasing, reducing, ameliorating a cancer and/or malignancy of any preceding aspect wherein the cancer and/or malignancy is detected or assessed in the subject using the methods of any preceding aspect.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, 1F, 1G, and 1H show the genomic landscape of transformed CTCL. FIG. 1A shows the Current Patient Cohort and Data Structure. FIGS. 1B and 1C shows the genomic landscape of transformed CTCL suggests high TMB with prognostic UV signature (unpublished data, manuscript in submission) FIG. 1B shows tumor mutation burden (TMB) in transformed CTCL (CTCL-MF) compared to 33 other cancer types in The Cancer Genome Atlas (TCGA). Analyses of mutation burdens of SBS, DBS and small indels showed transformed CTCL as a high tumor mutation burden cancer type second to skin melanomas (tumor types ordered by median SBS). FIG. 1C shows the mutation signature analyses using COSMIC v3 revealed significantly enriched SBS7a, SBS7b, and SBS7c signatures (prevail in UV-associated skin cancers) that are predictive of favorable survival in transformed CTCL, similar to previous findings in cutaneous melanoma. FIG. 1D shows overall survival probability of tCTCL patients classified according to high versus low SBS7 mutation signature (by sum of weighted contribution from SBS7a-d) and onset of LCT to time of death or last follow up. FIG. 1E shows the weighted contribution of SBS7a-d mutation signatures in Black/AA versus non-Black/AA patients. FIG. 1F shows a heatmap of cosine similarities between the mutational profile of each sample and COSMIC v3.2 mutational signature in Black/AA vs non-Black/AA patients, ranked by SBS7a-d. FIGS. 1G and 1H show Oncoplot of predicted driver genes by dNdScv and MutsigCV (1G) and most recurrently mutated pathway genes (1H) in tCTCL (n=70 samples). Each column represents a patient tumor sample. Somatic mutations, including missense (green), nonsense (red), in frame insertion (dark grey) and deletion (light blue), frame shift insertion (orange) and deletion (blue), splice site (yellow), multi hit (purple) and genes implicated in leukemia and lymphoma by manual curation (red asterisk) are depicted. TT (magenta), PP (gray). Recurrent mutations in >10% of the samples are depicted. The predicted driver genes belong to five groups: cell cycle, chromatin modification, cell motility, apoptosis, genes implicated in leukemia/lymphomagenesis (*asterisk) and other undefined (1G). Most recurrently mutated pathways are Hippo, Notch, RAS-RTK pathways and p53 (1H).

FIGS. 2A and 2B show that tCTCL exhibits distinct genomic gains and losses from those of SS/leukemic CTCL. FIG. 2A shows a composite plot of significant arm-level and focal SCNAs by WES, using GISTIC 2.0 to detect genes targeted by somatic copy-number alterations (SCNAs) that drive cancer growth. Transformed CTCL (tCTCL, top, n=55 samples with matched germline). SS/leukemic CTCL³⁶ (bottom, n=31 samples with matched germline). Each alteration is assigned a G-score (y-axis; frequency×amplitude). Amplifications (red, above the solid horizontal lines) and deletions (blue, below the solid horizontal lines) are plotted across the genome (x-axis). Select gene targets within the peak regions are depicted. Q-threshold=0.25. FIG. 2B shows the examination of 55 CTCL-associated genes reported in literature (mostly SS)¹⁹ and significantly differentially mutated genes in tCTCL (left, n=55 samples) vs SS/leukemic CTCL³⁶ (right, n=31 samples) (p<0.05 and q<0.25). Depicted are select candidate genes involved in DNA damage response, JAK/STAT, chromatin modification, T-cell activation, cell cycle, immune surveillance, PI3K, MAPK, T-cell differentiation, NFKB, cytoskeletal responding, T-cell migration), and significantly differentially mutated genes from unbiased comparison between tCTCL and SS cohorts.

FIGS. 3A, 3B, and 3C show a dissecting of the transformed CTCL TIME at single-cell resolution. FIG. 3A shows a single-cell profiling analytic workflow. 16 fresh skin biopsies were collected from 8 patients with CD4+ tCTCL (paired TT and PP lesions) for complementary 5′ scRNAseq and scV(D)Jseq. Single-cell V(D)J seq (10× chromium) detects the precise TCR α- and β-chain combination that defines each T-cell's TCR clonotype. “True malignant” cells (red)=cells with dominant TCR clonotypes, while “True benign” cells (green)=cells with non-dominant/polyclonal TCR clonotypes. FIG. 3B shows that malignant cells are further identified by inferred large-scale CNAs (inferCNV). The CNAs (red, amplifications; blue, deletions) are shown along the chromosomes for each cell. Two-thirds (⅔) of randomly selected non-dominant TCR/polyclonal CD4+ or CD8+ T-cells were input as the benign “Reference cells”. For the inferCNV observation group, the remaining ⅓ of “True benign” cells were “spiked in”, along with all clonal “Tue malignant” cells, and “Cells without TCR reads” (grey) that are positive for CD3 (i.e., the “Malignant suspect” cells, yellow) as the input cells. In the tCTCL TIME, “True malignant” cells by TCR clonality show malignant CNV patterns, while “True benign” cells show CNV neutral patterns. FIG. 3C shows uniform manifold approximation and projection (UMAP) of single-cell profile of 27,055 immune cells (dots), colored by malignant cell status (top left panel), with clear separation of malignant T-cells (red) from benign immune cell (green) clusters, patient ID (top right panel), and all malignant T-cells and benign immune cell types annotated in the TIME (bottom panel).

FIGS. 4A, 4B, 4C, 4D, 4E, and 4F show the malignant T-cell oncogenic program in tCTCL and pharmacologic inhibition of the OXPHOS and MYC in vitro. FIG. 4A shows gene set enrichment analysis (GSEA) comparing malignant T-cells in TT to benign CD4 T-cells showed significant enrichment of genes in OXPHOS, MYC, EMT (cellular plasticity/stemness) and E2F target pathways and down-regulation of IFN-γ, TNF-α, and IFN-α. The normalized enrichment score (NES, x-axis) reflects the extent of enrichment and allows comparison across gene sets. Listed pathways are ranked by their NES and colored by their significance. FIG. 4B shows GSEA. Comparison of malignant T-cells in TT to malignant T-cells in PP shows upregulation of genes in OXPHOS, MYC and E2F target pathways and down-regulation of IFN-γ and IFN-α pathways from PP to TT. FIG. 4C shows malignant T-cell oncogenic program DEGs. Scaled expression of select genes from the 55-genes tCTCL malignant T-cell oncogenic program (y-axis, rows) across the profiled cells (columns), malignant T-cells in TT (magenta) and benign CD4+ T-cells (green). Color bars to right denote significantly enriched pathways. Significant DEGs were filtered with a q value <0.05 and an absolute value of fold change (FC) >2 or <0.5 (Methods). FIG. 4D shows trajectories of the tCTCL TIME constituents in pseudotime by Monocle 3. UMAP for dimension reduction and visualization, including all cells previously annotated in FIG. 3C. Naïve CD4⁺ T cells in the graphical interface was designated as the root node, and the cellular trajectories in pseudotime were learned using the default parameters of Monocle 3 (learn_graph function). The learned trajectories reveal mono-directionality from benign T-cells to malignant T-cells in PP (grey) to malignant T-cells in TT (magenta) (right panel). A branch of the trajectory originating from naïve CD4+ T cells to malignant T cells was selected (choose_graph_segments function, left panel, purple branch), and the kinetics of the 55-gene malignant T-cell oncogenic program was plotted along pseudotime with select genes shown in the bottom panel (full list of genes in FIG. 8). Cell types as annotated in the scRNAseq dataset are dotted in colors (e.g., malignant T-cells in PP—grey, malignant T-cells in TT—magenta). CXCL13 (chemokine), SLC25A5 (OXPHOS), NME2 (MYC) showed coordinated up-regulation from benign T-cells to malignant PP, while EPCAM and TWIST (EMT/cellular plasticity) showed accentuation of gene expression at the end of the trajectory in tumors. Downregulation of HLA-A and HLA-B (MHC-I) occurred early at the bifurcation from benign to malignant PP in pseudotime. FIG. 4E shows violin plots of distribution of HLA-A, C, E, F gene expression in malignant T-cells in TT (magenta), malignant T-cells in PP (gray) and benign CD4 T-cells (green). **** denotes p<0.001. FIG. 4F shows T-cell lymphoma cell lines, Myla (MF), Jurkat (ATLL), HH (leukemic CTCL), MJ (ATLL) and Hu78 (SS). OXPHOS inhibitor (IACS-10759) apoptosis assay (left): indicated cell lines were seeded on 96-well plates and treated with 8 nM of IACS-010759 for 5 days. At day 5, cells were harvested and stained with Annexin V and PI following manufacture's protocol (Biolegend Cat #640914). Annexin V⁺PI⁺ population was gated on Singlet population using FlowJo 10 software. The data was normalized to vehicle control. The error bars represent the mean s.e.m. n=8 (MyLa and Jurkat); n=4 (HH, MJ, Hu78). MYC inhibitor (MYCi975) cell proliferation assay (right). Indicated cell lines were seeded on 96-well plates and treated with different dose of MYCi975 for 5 days. At day 5, cells were incubated with MTS reagent following manufacture's protocol (Promega Cat #G3580). Absorbance at OD490 nm was recorded and percentage of growth were normalized to vehicle control. Half maximal inhibitory concentration (IC50) was calculated based on curve fitting result using non-linear regression function of GraphPad Prism 8. The symbol represents the mean. The error bars represent the mean±s.e.m. n=12 (MyLa and Jurkat); n=8 (HH, MJ, Hu78).

FIGS. 5A, 5B, 5C, and 5D show the cellular crosstalk between malignant T-cells and the tCTCL TIME highlights MIF-CD74 interactions. FIG. 5A shows an overview of the statistically significant receptor-ligand interactions between malignant T-cells and macrophage/monocytes, B-cells, dendritic cells, endothelial cells and fibroblasts by integrating CellPhoneDB v2.0, a cell-cell communication informatics pipeline, with the single-cell RNAseq dataset (left; potential receptor-ligand pairs between interacting cell types denoted at top). Significance of p-values are indicated by circle size (−log 10 p-value, permutation test; Methods). Color indicates the log 2 means of the receptor-ligand pairs between 2 interacting cell types. Scale is shown to the right. Schematic (right) representing predicted top-ranking predicted ligand-receptor interactions between MIF in malignant T-cells and CD74 in macrophages/monocytes and B-cells in the tCTCL TIME. FIGS. 5B and 5C show MIF expression and MIF-CD74 co-localization by mIF immune profiling (80 core TMA). Lesion types: PP-NT (PP from non-transformed patients, n=16 tissue cores), PP-P (precursor PP from tCTCL patients, n=12 cores), PP-C(concurrent PP from tCTCL patients, n=12 cores), Transformed tumors (n=64 cores). Multi-layer TIFF images were exported from InForm (Akoya Biosciences) into HALO Image Analysis Platform (Indica Labs) for segmentation and quantitative analysis. FIG. 5B shows MIF (red), CD74 (sky blue), CD3 (green), Ki67 (dark blue), CD68 (yellow). Malignant CD4+ T-cell (CD3+ CD8− Ki67 high). Macrophages (CD68+ cells). MIF in malignant T-cells (left panel), MIF in malignant T-cells co-localizes with CD74 in macrophages (white arrow, middle panel). Boxplot depicting MIF-CD74 colocalization density (y-axis, count per mm2) against each lesion type. FIG. 5C shows MIF (red), CD74 (sky blue), CD3 (green), Ki67 (dark blue), PAX5 (yellow). Malignant CD4+ T-cell (CD3+ CD8− Ki67 high). B-cells (PAX5+ cells). MIF in malignant T-cells (left panel), MIF in malignant T-cells co-localizes with CD74 in B-cells (white arrow, middle panel). Boxplot demonstrating MIF-CD74 colocalization density (y-axis, cell count per mm2) against each lesion type. FIG. 5D shows TT (left panel) with dense infiltration of B-cells (PAX5+, yellow) between malignant CD4+ T-cells (CD3+ CD8− Ki67 high). A thick PP lesion from a non-transformed patient (middle panel) showing absence of PAX5+ B-cells in the TIME. Ki67 cells (dark blue) depict basal layer of the epidermis. Boxplot depicting B-cell density (y-axis, cell count per mm2) against each lesion type.

FIGS. 6A, 6B, and 6C shows dominant subclones in tCTCL show deregulation of ribosomal gene expression. FIG. 6A shows the identification of genetic subclones in malignant T-cells in each patient by partitioning hierarchical clustering trees (inferCNV, HMM subcluster mode, “qnorm” method). The CNAs (red, amplifications; blue, deletions) are shown along the chromosomes for each cell. Color bars to the right denote matched UMAP cluster annotation, patient ID and scaled expression. FIG. 6B shows UMAP plots of all malignant T-cells clustered by gene expression (left) and labeled by patient ID (right) reveal subclonal transcriptional heterogeneity in PT11 (clusters 1, 11, 12), PT 35 (clusters 0, 3, 9) and PT50 (clusters 4, 8). UMAP clusters and patient IDs (PTID) are color coded to the right. FIG. 6C shows DEG of the malignant T-cell subclusters in PT 35 (left) and PT11 (right) reveal dramatic upregulation of genes encoding ribosomal protein large and small subunits in the preponderant malignant T-cell subclones in these two patients with the worst clinical outcomes.

FIGS. 7A, 7B, and 7C show cutaneous tCTCL shows distinct malignant T-cell oncogenic prograrn from SS. FIG. 7A shows UMAP of malignant T-cells from 6 SS patients (Herrera cohort, patients SS1 to SS6) and malignant T-cells from 8 tCTCL patients in the current study (PT11, 35, 47, 50, 52, 53, 55, 56; each with PP and TT lesions). FIG. 7B shows UMAP of malignant T-cells from tCTCL patients (PP+TT, red) and SS patients (blue). FIG. 7C shows GSEA comparing malignant T-cells in tCTCL (PP+TT) vs malignant T-cells in SS shows significant upregulation of genes in TNF-α, MYC, EMT, OXPHOS and E2F target pathways and downregulation of genes in the IFN-α pathway. Normalized enrichment score (NES, x-axis). Listed pathways are ranked by their NES and colored by their significance. FIG. 7D shows violin plots of distribution of HLA-A, B, C gene expression in malignant T-cells in SS (blue), malignant T-cells in TT (magenta) and malignant T-cells in PP (gray). **** denotes p<0.001.

FIG. 8 shows the kinetics of 55-gene malignant T-cell oncogenic program in tCTCL (FIG. 4C) plotted along pseudotime (x-axis). Cell types dotted in colors (e.g., malignant T-cells in PP—grey, malignant T cells in TT-magenta, benign T-cells in remaining color schemes). Gene expression level based on scRNAseq data (y-axis) (FIG. 4D).

FIGS. 9A, 9B, 9C, and 9D show the effects various anti-cancer agents on malignant T cells. Cells are stained green with the Cytolight Rapid Green Dye—freely pass thru the membrane, however, once inside the dye is transformed into cell-impermeant probe transferred to daughter cells. Once the cells have been stained the dye is removed. Stains live cells. Then the Cytox Red is added and left in the media. Cytox Red moves inside the cells when the cell membrane is compromised. Assays were read at 0, 6, 12, 18 and 24 hrs. Each timepoint is analyzed separately for the designated concentration of Palbociclib (CDK4/6 inhibitor)(9A), Omacetaxine mepesuccinate (HHT)(9B), CX5461 (rRNA synthesis inhibitor)(9C), or BTZO-1 (MIF inhibitor)(9D).

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “biological sample” or “tissue sample” refers to any portion of biological material from a subject to be used in any of the methods or as a part of any of the compositions disclosed herein including, but not limited to, tissue biopsy, whole blood, serum, plasma, peripheral blood mononuclear cells, urine sample, lung lavage, sputum, saliva, cerebrospinal fluid, and fecal sample. The biological can include samples for normal and cancerous tissue. Sample may be obtained from any tissue a subject by any means known in the art (tissue resection, biopsy phlebotomy, core biopsy).

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular kit or method for the detection or diagnosis of a T or B cell malignancy is disclosed and discussed and a number of modifications that can be made to a number of molecules including the kit or method for the detection or diagnosis of a T or B cell malignancy are discussed, specifically contemplated is each and every combination and permutation of kit or method for the detection or diagnosis of a T or B cell malignancy and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Cutaneous T-cell lymphoma (CTCL) is a malignancy of skin-homing T cells. While cancer death rates have significantly declined for many common cancers in the past decade, there is a sobering under-representation of this success in rare cancers, and particularly in the vulnerable racial and ethnic minority groups. The lack of research tissue samples and clinical trials further compound this inequality. Several research groups have attempted to profile the genomic landscape of SS/MF by WES and WGS. While we have gained a wealth of information from these multi-institutional sequencing efforts, there is a lack of genome-level investigation of CTCL at disease transformation, with only 7 transformed MF WES reported to date. Similarly, recent advances in single-cell technologies have provided a high-resolution window into malignant and benign T-cell transcriptomics in a small number of SS and MF samples and revealed evidence of inter- and intra-tumoral heterogeneity. Malignant T-cells in skin show up-regulation of T cell activation, TCR ligation, and cell cycle progression transcripts as compared to those in the blood compartment. Despite these efforts, the paucity of investigation of CTCL tumor and immune microenvironment (TIME) at LCT or its relationship to precursor PP lesions contributes to inadequate knowledge concerning potential therapeutic targets for this deadly disease state.

To address this critical unmet need, we tackled the challenging tCTCL TIME by applying integrative multi-omics and multiplex immune profiling of skin biopsies from a rare cohort of 56 tCTCL patients. We comprehensively characterized the genomic landscape of tCTCL using tissue resource of similar size to other TCGA rare cancers, and established tCTCL as a high tumor mutation burden (TMB) cancer dominated by UV signatures that are prognostic for enhanced survival. Notably, Black/AA patients in our cohort show significantly lower contribution of UV signatures compared to the White patients. We identified predicted driver genes and recurrently altered pathways in Hippo, RAS/RTK and Notch, and showed that tCTCL in skin exhibits a distinct genomic chromosomal copy number variation (CNV) profile from SS/leukemic CTCL, which has important therapeutic implications. Using a combination of scRNAseq, scV(D)Jseq and CNV inference, we identified a unique malignant T-cell program with enrichment for oxidative phosphorylation (OXPHOS), cellular plasticity, upregulation of MYC and E2F activities, and down-regulation of MHC-I at transformation suggestive of immune escape. Furthermore, in vitro pharmacologic studies using novel small molecule inhibitors of OXPHOS (IACS-10759) and MYC (MYCi975) demonstrated potent anti-tumor activity. Immune profiling further revealed receptor-ligand interaction between MIF in malignant T-cells and CD74 in antigen-presenting cells (APCs). Notably, malignant T-cells in tCTCL demonstrated intra-tumoral genetic and transcriptional heterogeneity, with upregulation of ribosomal protein subunit gene expression in dominant subclones in patients with the poorest clinical outcomes. Collectively, our study provided the first comprehensive compendium of genomic alterations in CTCL at disease transformation, a conceptual blueprint for dissecting a complex lymphoma TIME, identified a tCTCL oncogenic program that exploits metabolic reprogramming, cellular plasticity and proliferation, and highlighted potential therapeutic vulnerabilities for this incurable rare cancer.

As noted above, Subgroup of patients develop large cell transformation and progress from indolent to aggressive lymphoma with a rapid decline in survival. Here, we investigated the genomic and transcriptomic landscape of transformed CTCL (tCTCL) using integrative approaches spanning whole exome sequencing (WES), parallel single-cell RNAseq and TCR seq and functional studies. We have applied a number of innovative single-cell analytic approaches on a unique cohort of large cell transformed CTCL patients, including parallel 5′scRNA-/scTCR-seq/copy number inference on same single-cells to separate malignant from benign T-cells in the transformed CTCL ecosystem. These approaches have thus far demonstrated success in dissecting a complex T-cell lymphoma tumor microenvironment, with the discovery of a 77-gene malignant T-cell program and intra-tumoral heterogeneity by gene expression and CNV inference in tCTCL with upregulation of ribosomal synthesis and translation signature in the most expanded malignant T-cells clones in tCTCL.

In one aspect, disclosed herein are methods of detecting malignant cells in a tumor microenvironment or methods of diagnosing a cancer type in a subject, said method comprising obtaining a tissue sample (such as, for example, tissue samples comprising T cells and B cells including, but not limited to B cell and T cell tumor samples), performing whole exome sequencing (WES), parallel single-cell RNAseq, single cell TCRseq and/or copy number variation (CNV) interference assay on the sample; wherein the pattern of increase or decrease in gene expression and/or the presence of mutations relative to a control indicates that the subject does or does not have a malignancy.

It is shown herein that changes in gene expression and mutations associated with malignancies are particularly prevalent in genes related to Oxidative phosphorylation, MYC targets V1, MYC targets V2, epithelial mesenchymal transition, E2F targets, xenobiotic metabolism, coagulation, WNTb catenin signaling, cholesterol homeostasis, spermatogenesis, estrogen response late, and DNA repair, Thus, disclosed are methods of detecting malignant cells in a tumor microenvironment, or methods of diagnosing a cancer type in a subject wherein a malignancy is indicated by mutations in genes related to Oxidative phosphorylation, MYC targets V1, MYC targets V2, epithelial mesenchymal transition, E2F targets, xenobiotic metabolism, coagulation, WNTb catenin signaling, cholesterol homeostasis, spermatogenesis, estrogen response late, and DNA repair. In one aspect, disclosed herein are methods of detecting malignant cells in a tumor microenvironment or methods of diagnosing a cancer type in a subject, wherein the cancer is Cutaneous T-cell lymphoma (CTCL) and the presence of a malignancy is indicated by an increase in 5 or more of the genes selected from the list consisting of CD9, CD74, COPA, ADDD, RAB25, LSR, CLDN7, EPCAM, TWIST1, LGALS3, S10A6, SLC25A5, PIM3, 1L22, PTHLH4, CCR7, AHR, CORO1B, ROMO1, MIF, NME2, NDUFB2, SRM, LAIR2, HTRA1, PAGE5, IGFBP2, AREG, XCL2, and XCL; and/or a decrease in the expression of 5 or more of the genes consisting of RGCC, ANXA1, FOS, ZFP36, HLA-DPB1, HLA-DPA1, HLA-DRB1, SAT1, FTH1, HSPA1B, DNAJB1, GLIPR1, IL10RA, SELL, TXNIP, ARID5B, RHOH, BTG1, TPT1, HLA-B, HLA-A, HLA-F, TNFRSF4, IL2RA, and GBP5. In one aspect the malignancy is detected by the colocaization of one or more genes with increased expression (such as, for example, MIF and CD74, CD74 and COPA, CD74 and ADD, HLA-B and KIR3DL2, and/or HLA-C and FAM3C)

It is understood and herein contemplated that the relevant assays detect different aberrations. Thus, disclosed herein are disclosed herein are methods of detecting malignant cells in a tumor microenvironment or methods of diagnosing a cancer type in a subject, wherein the cancer malignancy is mycosis fungoides (MF), wherein at least on assay comprises copy number variation (CNV), and the presence of the malignancy is indicated by an increase in the expression of four or more of the genes selected from the group consisting of SYCP1, TM4SF1, AHR, NDUFB2, TWIST1, LGALS3, RBBP6, and NDUFA7 and a decrease of six or more of the genes selected from the group consisting of PRAMEF, PCDHGB, PMS2P1, RECQL4, IFNA, CDKN2, NOTCH1, HRAS, POLE, AKT1, DEFB, and SOCS7. Similarly, disclosed herein are methods of detecting malignant cells in a tumor microenvironment or methods of diagnosing a cancer type in a subject, wherein the cancer malignancy is mycosis fungoides (MF) and the presence of the malignancy is indicated by a mutation in three of more of the genes selected from the group consisting of FSIP2, TEKT4, PCDH15, TP53, STAT3, JAK3, KMT2C, CREBBP, ARID1A, TRRAP, KMT2D, PLCG1, and CDKN2A. Also disclosed herein are disclosed herein are methods of detecting malignant cells in a tumor microenvironment or methods of diagnosing a cancer type in a subject, wherein the cancer malignancy is leukemic CTLC; wherein at least on assay comprises copy number variation (CNV), and the presence of malignancy is indicated by an increase in the expression of CARD11 and/or MUC16 and a decrease in the expression of 5 or more of the genes selected from the group consisting of ARID1A, TNFAIP3, CDKN2A, DAPL1, WT1, ATM, CDKN1B, SOCS2, RB1, and STK11.

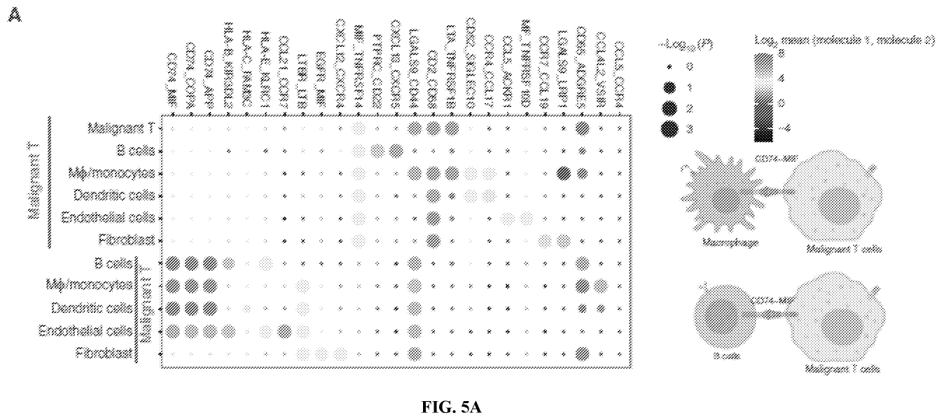

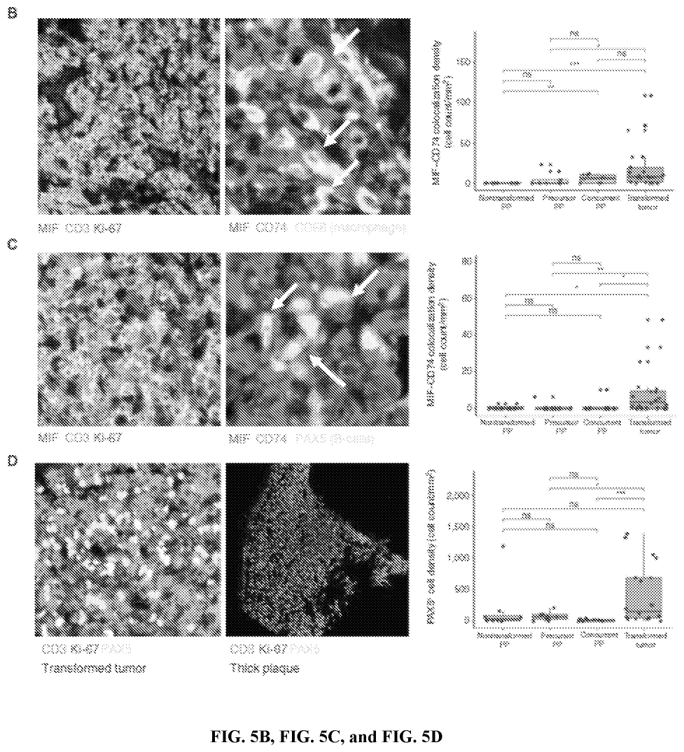

It is also shown herein that co-localization of genes in a cell can indicate a malignancy and/or a more the aggressiveness of a cancer (i.e, a poor prognosis). For example, co-localization of MIF and CD74, CD74 and COPA, CD74 and ADD, HLA-B and KIR3DL2, and/or HLA-C and FAM3C indicates a poor prognosis (see FIG. 5A). Thus, in one aspect, disclosed herein are methods of detecting a malignancy or assessing the severity of a cancer comprising obtaining a tissue sample (such as, for example, tissue samples comprising T cells and B cells including, but not limited to B cell and T cell tumor samples, as well as tissue, tissue samples comprising dendritic cells monocytes, and endothelial cells), performing whole exome sequencing (WES), parallel single-cell RNAseq, single cell TCRseq and/or copy number variation (CNV) interference assay on the sample; wherein a poor prognosis (i.e., a more sever cancer or more malignant cancer) is indicated by the co-localization of MIF and CD74, CD74 and COPA, CD74 and APP, HLA-B and KIR3DL2, and/or HLA-C and FAM3C. In one aspect, the method can further comprise treating the cancer with a MIF inhibitor.

The disclosed methods of detecting a cancer or malignancy, diagnosis a cancer or malignancy or detecting a cancer type can be used to in the detection and/or diagnosis of any disease where uncontrolled cellular proliferation occurs such as cancers. A representative but non-limiting list of cancers that the disclosed compositions can be used to treat is the following: lymphoma, B cell lymphoma, T cell lymphoma (including, but not limited to cutaneous T cell lymphomas), mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer (including, but not limited to TNBC), and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. Thus, in one aspect, disclosed herein are disclosed herein are methods of diagnosing or detecting a cancer or cancer type of any preceding aspect, wherein when a diagnosis of cancer is made or a cancer is detected or a cancer type is determined (such as, for example, the detection and or diagnosis of Cutaneous T-cell lymphoma (CTCL), melanoma, and/or breast cancer (including, but not limited to TNBC), the method further comprises treating the subject comprising administering to the subject one or more anti-cancer agents (such as, for example, an OXPHOS, Myc, MIF, and/or CDK4/6 inhibitor (including, but not limited to Palbciclib).

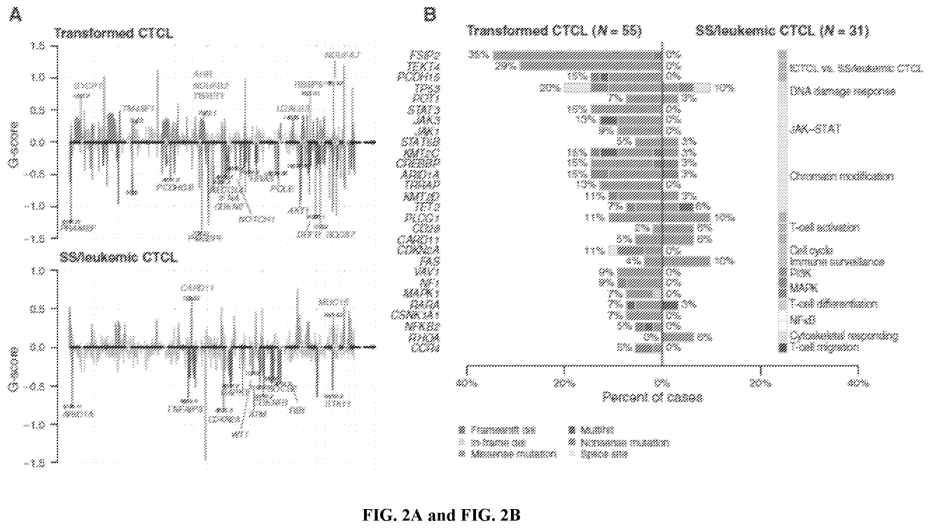

It is noted herein that the present methods of detection and/or diagnosis show different results between blood cancers and cutaneous cancers (such as melanoma or a breast cancer (including, but not limited triple negative breast cancer (TNBC)) as evidenced by FIGS. 2A and 2B. Furthermore, the detection of a cancer, cancer type, or a particular gene signature can direct the practicing physician to the appropriate treatment for the subject (i.e., which drugs will work). For example, the disclosed methods can be used to detect breast cancer (including, but not limited to TNBC) and suggest the use of a CDK4/6 inhibitor, such as Palbociclib alone or in combination with an OXPHOS, Myc, MIF inhibitor to treat the breast cancer.

### 1. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

Also disclosed herein are kits for diagnosing a cancer in a subject or detecting a malignancy in a subject comprising primers and/or probes for the detection of mutations or the expression of five or more genes selected from the group consisting of CD9, RAB25, LSR., CLDN7, EPCAM, TWIST1, LGALS3, S100A6, SLC25A5, PIM3, 1L22, PTHLH, CCR7. AHR, CORO1B, ROMO1, MIF, NME2, NDUFB2, SRM, LAIR2, HTRA1, PAGE5, IGFBP2, AREG, XCL2, XCL1, RGCC, ANXA1, FOS, ZFP36, HLA-DPB1, HLA-DPA1, HLA-DRB1, SAT1, FTH1, HSPA1B, DNAJB1, GLIPR1, IL10RA, SELL, TXNIP, ARID5B, RHOH, BTG1, TPT1, HLA-B, HLA-A, HLA-F, TNFRSF4, IL2RA, GBP5, FSIP2, TEKT4, PCDH15, TP53, STAT3, JAK3, KMT2C, CREBBP, ARID1A, TRRAP, KMT2D, PLCG1, CDKN2A, SYCP1, TM4SF1, AHR, NDUFB2, TWIST1, LGALS3, RBBP6, NDUFA7, PRAMEF, PCDHGB, PMS2P1, RECQL4, IFNA, CDKN2, NOTCH1, HRAS, POLE, AKT1, DEFB, SOCS7, CARD11, MUC16, ARID1A, TNFAIP3, CDKN2A, DAPL1, WT1, ATM, CDKN1B, SOCS2, RB1, and STK11.

### 2. Method of Treating Cancer

The disclosed kits, methods, can be used to in the treatment of any disease where uncontrolled cellular proliferation occurs such as cancers. A representative but non-limiting list of cancers that the disclosed compositions can be used to treat is the following: lymphoma, B cell lymphoma, T cell lymphoma (including, but not limited to cutaneous T cell lymphomas), mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer (including, but not limited to TNBC), and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

In one aspect, any of the methods of detecting malignant cells in a tumor microenvironment as disclosed herein, methods of assessing the aggressiveness of a cancer as disclosed herein, methods of detecting a cancer as disclosed herein, or methods of diagnosing a cancer type in a subject disclosed herein, can comprise obtaining a tissue sample (such as, for example, tissue samples comprising T cells and B cells including, but not limited to B cell and T cell tumor samples), performing whole exome sequencing (WES), parallel single-cell RNAseq, single cell TCRseq and/or copy number variation (CNV) interference assay on the sample; wherein the pattern of increase or decrease in gene expression, co-localization of certain genes (e.g., MIF and CD74, CD74 and COPA, CD74 and APP, HLA-B and KIR3DL2, and/or HLA-C and FAM3C). and/or the presence of mutations relative to a control indicates that the subject has a malignancy, indicates the severity of the cancer, detects the cancer or provides for a diagnosis of cancer; said methods further comprise treating a detected malignancy by administering to a subject an anti-cancer agent such as, for example, an OXPHOS, Myc, MIF, and/or CDK4/6 inhibitor (such as, for example, Palbociclib). In other words, disclosed herein are methods of treating, inhibiting, decreasing, reducing, ameliorating and/or preventing a cancer and/or malignancy in a subject (such as, for example, a T-cell lympohoma) comprising administering to the subject an OXPHOS, Myc, MIF, and/or CDK4/6i inhibitor (such as, for example, Palbociclib). In one aspect, disclosed herein are any of the methods of treating, inhibiting, decreasing, reducing, ameliorating a cancer and/or malignancy of any preceding aspect wherein the cancer and/or malignancy is detected or assessed in the subject using any of the methods disclosed herein.

The anti-cancer agent can comprise any anti-cancer agent known in the art including, but not limited to antibodies, tumor infiltrating lymphocytes, checkpoint inhibitors, dendritic cell vaccines, anti-cancer vaccines, immunotherapy, and chemotherapeutic agents. In one aspect, the anti-cancer agent can include, but is not limited to an OXPHOS, Myc, MIF, and/or CDK4/6i inhibitor (such as, for example, Palbociclib), Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil--Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil--Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil--Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil--Topical), Fluorouracil Injection, Fluorouracil--Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, IBRANCE® (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), mogamulizumab, MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), OPDIVO (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, ,Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil--Topical), Topotecan Hydrochloride, Toremifene, Torisel (Temsirolimus), Tositumomab and Iodine 1131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Also contemplated herein are chemotherapeutics that are checkpoint inhibitors, such as, for example, PD1/PDL1 blockade inhibitors and/or CTLA4/B7-1 or 2 inhibitors (such as, for example, PD-1 inhibitors lambrolizumab, OPDIVO® (Nivolumab) (BMS-936558 or MDX1106), CT-011, MK-3475, KEYTRUDA® (pembrolizumab), and pidilizumab; PD-L1 inhibitors MDX-1105 (BMS-936559), MPDL3280A, MSB0010718C, TECENTRIQ® (Atezolizumab), IMFINZI® (Durvalumab), and BAVENCIO® (Avelumab); and CTLA-4 inhibitors YERVOY (ipilimumab) (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96,- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, BI754111, Sym022, FS118, MGD013, and Immutep).

In some aspects, a combination of therapeutic agents can be used to treat the cancer. For example, a CDK4/6 inhibitor and an OXPHOS inhibitor can be used together which can have beneficial effects beyond any therapeutic synergy including lowering toxic effects of a drug by allowing a lower dosage to be used. In this way the use CDK4/6 inhibitor (such as, Palbociclub) with an OXPHOS inhibitor can result in a lower effective dosage of the OXPHOS inhibitor being needed.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Cutaneous T-cell lymphomas (CTCLs) are a group of non-Hodgkin lymphoma of skin-homing T cells. The most common form of CTCL, mycosis fungoides (MF, 50-60% of CTCL), typically behaves as an indolent lymphoma at presentation and is characterized by cutaneous manifestations of patch/plaque or tumor lesions, while Sezary syndrome (SS, 2-3% of CTCL) is a leukemic form of CTCL with circulating malignant T-cells in the peripheral blood and clinically manifests as generalized erythroderma. Transformation of CTCL occurs when there is a histopathologic change from neoplastic small-medium lymphocytes to large, blast-like T-cells. Similar to Richter's transformation in chronic lymphocytic leukemia/small lymphocytic lymphoma, large cell transformation (LCT) in CTCL heralds immediate transition to aggressive clinical behavior, rapid decline in survival (<2 years in ˜50-60% of patients) and resistance to multiple forms of therapy. The primary site of detectable transformation is usually in the skin (>92%). Major advances have been made in the treatment of advanced stage disease with the FDA approval of mogamulizumab (anti-CCR4) as a breakthrough therapy for CTCL in 2018. Despite the remarkable success compared to other forms of systemic therapy for advanced stage CTCL, majority of the patients did not respond to mogamulizumab monotherapy (MAVORIC trial³). The ORR was 25.3% at 4 months, with a median PFS of 7.8 months. Furthermore, responses were compartmentalized, with responses in blood and skin seen in 49.2% and 27.4% of patients⁴, respectively, and LCT was one of the exclusion criteria in this trial. The MAVORIC trial thus highlights a critical need to identify therapeutic targets for advanced stage disease in the skin compartment as either monotherapy or combination therapy with mogamulizumab.

The lack of effective therapies for advanced stage CTCL partly stems from our insufficient understanding of its disease biology. Several research groups have attempted to profile the genomic landscape of CTCL by WES⁶⁻¹⁴. While we have gained a wealth of information from these multi-institutional sequencing efforts, a notable limitation of these studies is the lack of genome level investigation of CTCL at LCT, with only 5 LCT and 2 samples described as “CTCL-NOS with large cell morphology” analyzed to date. Recent single cell profiling of one Sezary patient using parallel scRNA-scTCRseq revealed monoclonal T-cells with intratumoral heterogeneity by gene expression clustering. Likewise, profiling of skin CTCLs of mixed immunophenotypes by scRNAseq (5 patients; CD4+, CD8+ and CD4-CD8-MF) revealed inter-tumoral heterogeneity, signature of highly proliferating T-cells, intra-tumoral heterogeneity in one patient, and background immune effector and exhaustion programs, though the lack of complementary strategies for defining malignancy poses potential challenge in data interpretation and likely masks important malignant T-cell features in a heterogeneous immune tumor microenvironment, as in bulk RNAseq studies. Importantly, there is a paucity of investigation of CTCL at transformation that contributes to inadequate knowledge concerning potential therapeutic targets for this deadly disease at its most aggressive state.

To address these limitations, we tackled the challenging lymphoma tumor microenvironment by applying integrative multi-omics approaches and immune profiling of skin biopsies from a unique cohort of transformed CTCL patients. First, we comprehensively characterized the genomic landscape of tCTCL and established tCTCL as a high TMB cancer dominated by UV signatures that are prognostic for survival. We identified predicted driver genes and recurrently mutated pathways in Notch, Hippo, Ras and Wnt. We further showed that tCTCL in the skin compartment exhibits a distinct genomic CNV profile from the leukemic form of CTCL, which has important implications for identifying druggable targets in the skin. Using complementary scRNAseq, scTCRseq and CNV inference, we identified a unique malignant T-cell signature with metabolic reprogramming toward oxidative phosphorylation, cellular plasticity, upregulation of Myc and E2F activities, and immune escape through down-regulation of MHC-I. In vitro pharmacologic studies using novel small molecule inhibitors of OXPHOS (IACS-10759) and MYC (MYCi975) in CTCL cell lines supported the malignant T-cell signature by demonstrating potent anti-tumor activity. Notably, intra-tumoral heterogeneity at the genetic and expression level shows deregulation of ribosomal proteins in dominant malignant T-cell subclones in patients with poorest clinical outcome. Collectively, our studies provided a conceptual blueprint for dissecting a complex lymphoma immune tumor microenvironment, identified a transformed CTCL oncogenic program that exploits cellular plasticity, proliferation and metabolic reprogramming, and highlighted potential therapeutic targets for this incurable cancer.

### 1. Example 1: Cutaneous T-Cell Lymphoma Exploits an Oncogenic Program with Metabolic Reprogramming, Cellular Plasticity and Ribosomal Protein Deregulation at Large Cell Transformation Results

### a) Results

(1) tCTCL is a High TMB Cancer and Presence of UV Signatures is Prognostic for Survival at LCT

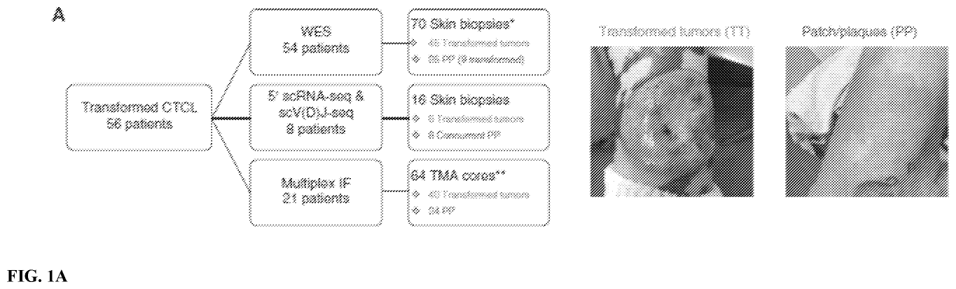

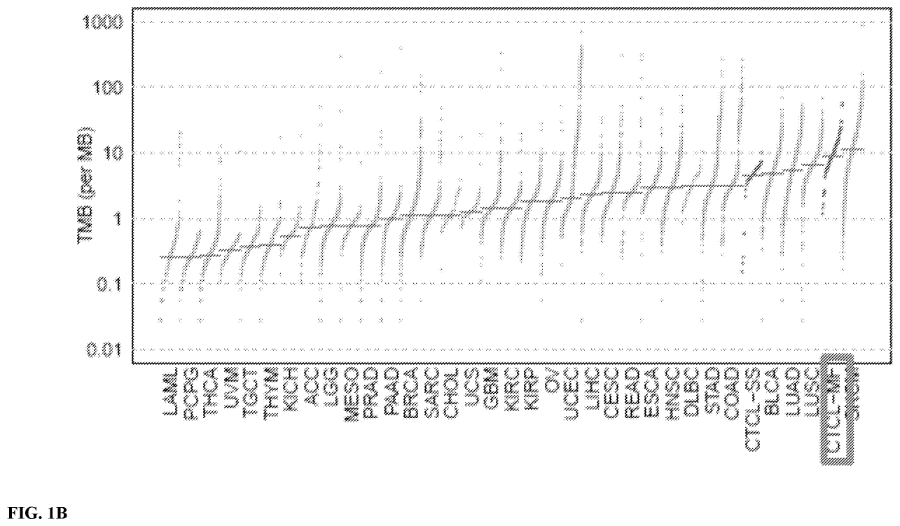

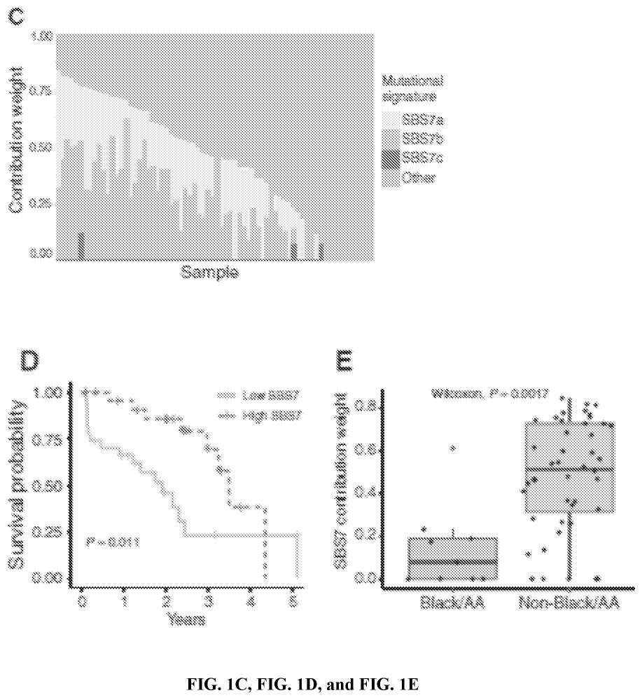

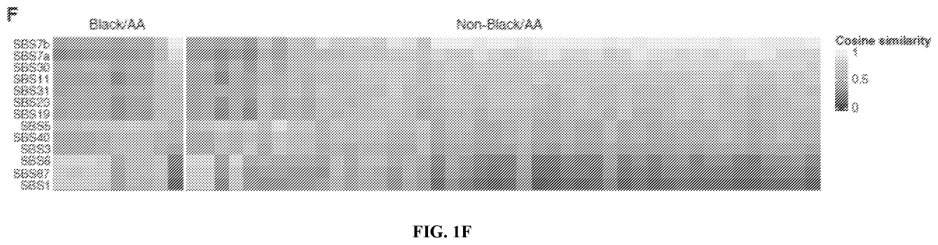

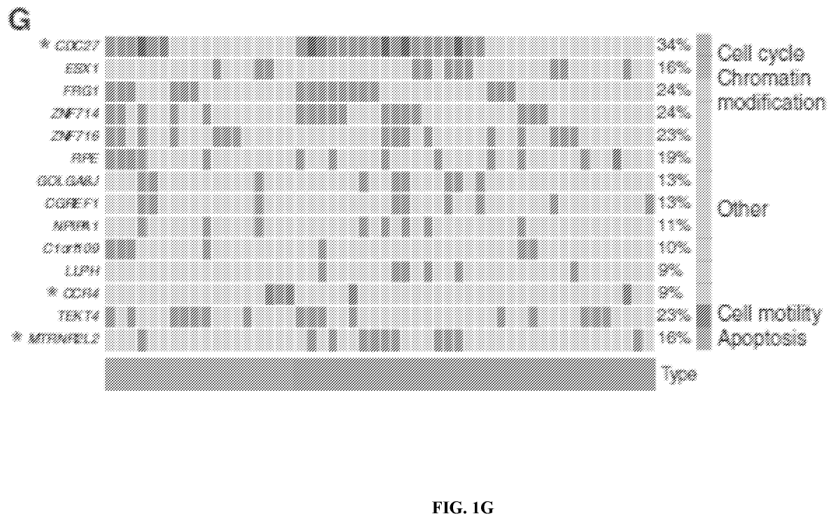

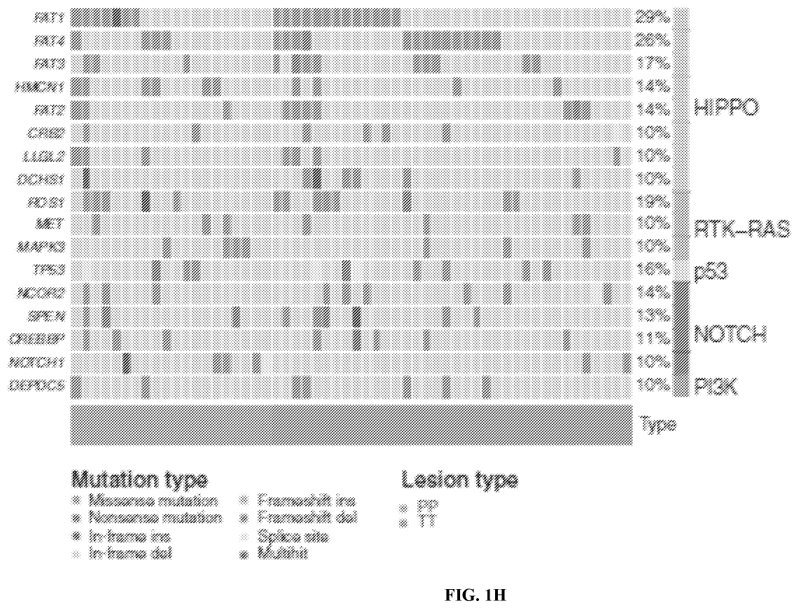

We collected a unique cohort of 56 patients with biopsy-proven tCTCL (53 transformed MF, 3 SS/overlap MF-SS with transformed tumors in skin) who were seen and/or treated between 2014 to 2020. Of the 56 patients, there were 38 males (68%), 18 females (32%), and 17 patients belonging to racial/ethnic minority groups (30%). Clinical re-staging was performed at the time of LCT. The median time from initial MF diagnosis to LCT was 25.3 months (range 0-252.5 months), median time from LCT to death or last follow-up was 20.4 months (range 1.0-61.3 months), and 26 patients were deceased (46.4% of the cohort). Available biopsies from these patients were carefully annotated with clinical data elements and processed for multi-omics profiling, including WES, 5′ scRNAseq, scV(D)Jseq and multiplex immunofluorescence (mIF) immune profiling (FIG. 1A).

We first sought to explore the genomic landscape of tCTCL and collected 70 skin biopsies from 54 patients with confirmed LCT in skin for WES [FIG. 1A; 45 transformed tumors (TT), 9 patch/plaque lesions (PP) with LCT, and 16 concurrent or precursor PPs to TT]. Of the 54 patients sequenced, 41 had matched germline normal samples, and 17 had paired PP and TT. Somatic single nucleotide variants (SSNVs) and small indels were called by comparing tumor and PP to matched normal germline or panel of normal (n=13 patients). We identified a median of 290 mutations per sample with a dominance of missense mutations (97.1%) and C>T transitions (65.8%) that is comparable to those described in SS and MF. We measured the tumor mutation burden (TMB) in all samples with matched germline and compared this to 33 other cancers profiled in The Cancer Genome Atlas (TCGA) and two independent cohorts of SS (Wang and Choi cohorts). Analyses of mutation burdens of SSNVs and small indels in tCTCL demonstrated a TMB that is exceeded only by lung SCC and cutaneous melanoma and significantly higher than SS (FIG. 1B; tumor types ordered by median TMB).

To comprehensively interrogate the operative mutational processes in tCTCL, we next catalogued the repertoire of mutational signatures with reference to the COSMIC mutational signatures (v3.2). At the sample level, we used deconstructSigs to analyze the weights of mutation signatures and showed that UV signatures SBS7a and SBS7b carried the maximum weight (FIG. 1C;). Similarly, we implemented MutationalPatterns and demonstrated highest cosine similarity to signatures SBS7a and SBS7b, with no significant difference between PPs and tumors. At the cohort level, novel mutational signatures derived by non-negative matrix factorization (NMF) methods showed best match to SBS7b and SBS6. The predominance of UV signatures has been reported in SS/MF and other sun-protected cancers, and Signature 7 has been shown to be prognostic for favorable outcome in cutaneous melanoma. We performed survival analysis and showed that the sum of weighted signatures SBS7a, SBS7b, SBS7c and SBS7d are also prognostic for favorable survival from the time of LCT (FIG. 1D). Importantly, Black/AA patients in our cohort show significantly lower contribution of UV signatures compared to the white patients (FIG. 1E), with enrichment of SBS6, SBS1 and SBS87 in patients with low UV signatures (FIG. 1F). Large-scale epidemiology studies showed that Black/AA patients with MF demonstrated poorer survival than white patients. We also observed a survival gap between Black/AA and white patients in this cohort.

(2) Exome-Based Sequencing Identifies Driver Events and Key Oncogenic Pathways in tCTCL

We next sought to detect exome-based driver events in tCTCL utilizing two mutation-based computational tools, dNdScv and MutSigCV, to enhance the coverage of our analysis. dNdScv is a statistical model based on refined dN/dS while factoring variation of mutation rate, and MutSigCV assesses mutation significance as a function of gene size, trinucleotide context and background mutation frequency for highly recurrent mutations. When both dNdScv and MutsigCV were performed on the same dataset, a total of 20 driver genes were identified, including CCR4 (chemokine receptor), FRG1 (chromatin modifying), TEKT4 (cell motility), CDC27 and ESX1 (cell cycle), MTRNR2L2 (anti-apoptosis) and other novel genes previously not implicated in CTCL (FIG. 1G). Notably, we observed CCR4 mutations concentrated at the C-terminus, similar to other gain-of-function CCR4 C-terminus mutations seen in adult T-cell leukemia/lymphoma (ATLL) and SS. We subsequently performed analyses of recurrently mutated pathways and showed top ranking mutations in Hippo (FAT1, FAT4), Notch (NCOR, SPEN, CREBBP), RAS (ROS1, MEK, MAPK3) pathways and p53 (FIG. 1H). Importantly, Hippo and Notch are key signaling pathways known to be essential for organ size control in development, stemness and tumor suppression, and deletion of FAT1 has been shown to promote stemness and metastasis through epithelial to mesenchymal transition in cancer. We also stratified the WES samples by CD30 expression (<:1%, >1% to <10%, 10-50%, >50%) but did not detect significant differences in overall survival, TMB, UV signatures, driver or top-ranking pathway genes.

(3) Cutaneous tCTCL Exhibits Distinct Genomic Gains and Losses from Those of SS/Leukemic CTCL

Work in SS reported distinct chromosomal copy number variation patterns, with characteristic recurrent alteration in genes such asARID1A, CDKN2A, CDKNIB, ZEB1, DNMT3A, PLCG1, TP53, PDCD1 and CARD11. We next explored the tCTCL genome for candidate somatic copy-number alterations (SCNAs). Using GISTIC 2.0, an algorithm that detects genes targeted by SCNVs that drive cancer growth, we mapped 42 recurrently deleted loci (33 with Q-values <0.005) and 28 recurrently amplified loci (9 with Q-value <0.005) across the genome (Q threshold 0.25; FIG. 2A). Recurrent deletions were seen in the PRAME family of genes (cancer testis antigen/stemness), clustered Protocadherin family, Defensins (DEFB109P1 and DEFB130), RECQL4 (genomic stability and chromatin modifying), CDKN2A (cell cycle), SOC6 and SOC7 (cytokine signaling, JAK/STAT) and POLE (DNA repair), while recurrent amplifications were seen in TWIST1 (cellular plasticity/stemness), NDUFB2 and NDUFA7 (OXPHOS), RPLs and RPSs (ribosomal protein subunits), LGALS3 (Galectin3/RAS), AHR (T-cell differentiation), CARD11 (T-cell activation) and SYCP1 (cancer testis antigen). We also performed GISTIC analysis on available SS/leukemic CTCL samples (FIG. 2A). While tCTCL and SS/leukemic CTCL shared conserved alterations in genes such as CARD21 and CDKN2A, the global genomic profiles between the two entities are strikingly different, with more prominent G-scores seen in tCTCL (FIG. 2A).

Recent WES meta-analysis of predominantly SS patients described 55 putative CTCL driver genes involving pathways that affect chromatin remodeling, immune surveillance, MAPK, NF-κB and PI-3-kinase signaling. We therefore investigated these candidate genes in tCTCL and compared to a publicly available SS/leukemic CTCL dataset from this meta-analysis study (Choi cohort). Interestingly, while both cohorts show similarly recurrent mutations in genes such as STAT5B, POT1, TET2, and CARD11, tCTCL demonstrated drastically more recurrent mutations in the JAK-STAT pathway genes (JAK1, JAK3, STAT3), key chromatin modifying genes (KMT2C, KMT2D, ARID1A, CREBBP), TP53 and CDKN2A (FIG. 2B), indicating more global dysregulation in genome-wide topology and gene expression in tCTCL. Recurrent mutations described as hotspot mutations in T-cell and/or NK T-cell lymphomas such as CD28^{F511}, JAK3^{A573V} and STAT5B^{N642H} were conserved in tCTCL, with the latter showing therapeutic vulnerability to JAK-STAT inhibitors. An unbiased comparison of the tCTCL and SS cohorts identified 116 significantly differentially mutated genes, including novel genes such as FSIP2, TEKT4 and Protocadherin 15 that are mutated only in tCTCL but not SS (p<0.05,Q<0.25). Dissimilarity in the genomic landscape of transformed T-cells in skin versus leukemic T-cells in blood provides opportunities to exploit differential or synergistic therapeutic vulnerabilities in the two body compartments at advanced stage disease.

### (4) Dissecting the Transformed CTCL TIME at Single-Cell Resolution

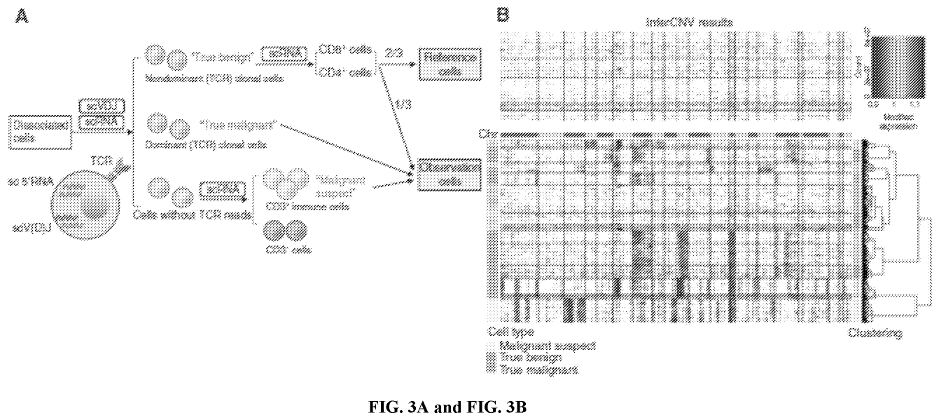

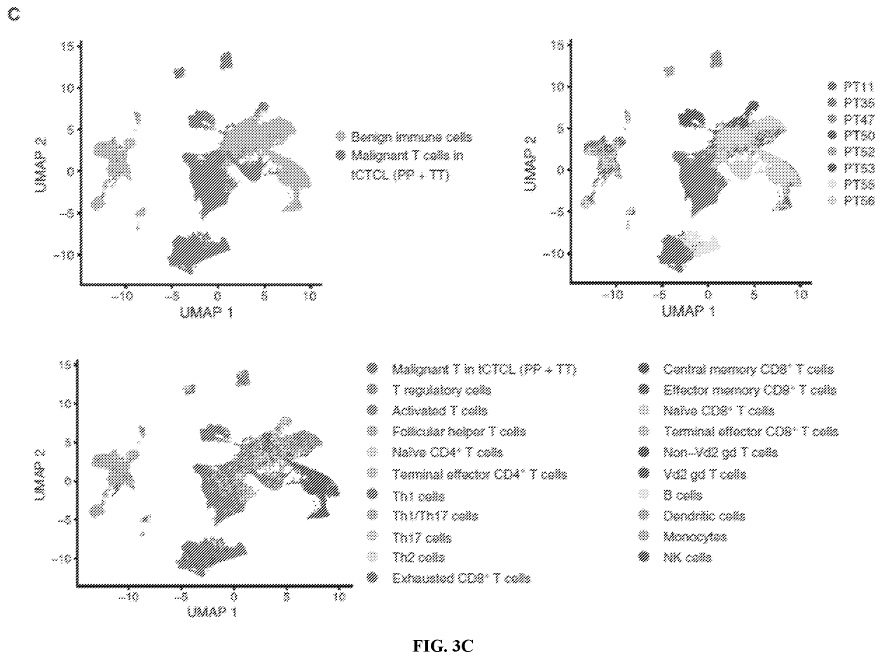

To interrogate the tCTCL tumor ecosystem, we next sought to profile the tCTCL TIME at single-cell resolution. We collected 16 fresh skin biopsies from 8 tCTCL patients, each with paired PP and TT, and profiled 34,912 cells using complementary 5′ scRNAseq and scV(D)Jseq strategies (10× Chromium) (FIG. 1A, FIG. 3A). Single cell V(D)Jseq identifies the precise TCR α- and β-chain clonotype combination in individual T-cells and allows linkage of clonotype information to the whole transcriptome by complementary 5′ scRNAseq. Despite the advantage of scV(D)Jseq in identifying clonal TCR, it is still imprecise due to “dropout” read, a well-known phenomenon in both scRNAseq and scV(D)Jseq datasets. To tackle this issue, we sought to separate malignant from benign T-cells by two complementary profiling strategies. First, we defined true malignant and true benign T-cells by scV(D)J seq (FIG. 3A). Next, we distinguished malignant cells by patterns of inferred SCNAs using the 5′ scRNAseq data (inferCNV, FIG. 3B, Methods). In 7 of 8 patients, we observed a dominant TCR clonotype of paired α- and β-chain sequences, consistent with TCR monoclonality in cutaneous tCTCL. Concurrent PP and TT lesions from the same patient also shared the same TCR clonotype. Interestingly, even though some lesions appear to have multiple “dominant” clonotypes (e.g. PT35T, clonotypes 1-7), careful inspection reveals the same TCR α-chain (TRA: CAVGDAGGTSYGKLTF (SEQ ID NO: 1)) and -chain sequences (TRB: CASSRQGGSGNTIYF (SEQ ID NO: 2) or CASSRLGGSGNTIYF (SEQ ID NO: 3)) across all 7 clonotypes. Two TRB amino acid sequences that differed by single amino acid were observed in the most dominant clonotype (TRA: CAVGDAGGTSYGKLTF (SEQ ID NO: 1); TRB: CASSRQGGSGNTIYF (SEQ ID NO: 2); TRB:CASSRLGGSGNTIYF (SEQ ID NO: 3)), perhaps due to somatic hypermutation. Cells exhibiting unpaired TRA and TRB (e.g. PT35 clonotypes 4, 6, 7) were present, consistent with “dropout” reads in either TCR α- or β-chain expression, respectively. Due to the observed TCR read dropout, a second malignancy detection method using inferCNV was implemented.

An important basis for inferCNV is the requirement for reference “normal” cells, ideally of the same cell type, and a separate group of “observation” cells for testing. Here, we selected cells harboring the non-dominant/polyclonal TCR clonotypes from scV(D)J profiling as “true normal”. We further split the “true normal” cells and input ⅔ of these cells into the inferCNV reference cell group (FIGS. 3A and 3B). For the observation group, we “spiked in” the remaining “true normal” cells and input all cells with dominant TCR clonotype (i.e. “true malignant”), as well as all CD3+ T-cells with TCR dropout as “malignant suspect”. We hypothesized that malignant T-cells would demonstrate malignant CNV patterns and form distinct clusters from normal T-cells with neutral CNV pattern. Indeed, we observed a striking dichotomy in chromosomal large-scale CNV pattern between malignant versus normal cells, with increased patient-specific CNVs observed in malignant T-cells (FIG. 3B). UMAP clustering of all cells generated 17 clusters, with distinct separation between malignant T-cells harboring positive CNV patterns and benign T-cells with neutral CNV patterns (FIG. 3C). As in other cancer types, tCTCL demonstrates significant inter-patient heterogeneity by gene-expression, with malignant T-cells clustering by patient of origin, and normal T-cells from different patients clustering together, indicating consistency of the background immune infiltrate.

Notably, of the 3 samples that lacked dominant TCR clonotypes (PT53 TT, PT55 PP, PT55 TT): PT53 TT showed CNV neutral patterns in all cells, consistent with lack of viable tumor cells in that sample, while PT55 PP and PT55 TT showed cell populations with striking malignant CNV patterns, consistent with presence of scV(D)Jseq drop out. Therefore, our approach demonstrates that the combination of scV(D)Jseq and CNV evaluation provides a more robust methodology for distinguishing malignant from benign reactive T-cells in T-cell lymphoma single-cell studies. After separating malignant T-cells from the tCTCL TIME, the remaining CD45/PTPRC+ benign immune cell populations were annotated by known marker genes (FIG. 3C) and SingleR, a computational algorithm that performs unbiased cell type recognition by referencing transcriptomic datasets of pure cells types (Monaco immune database. Using these two approaches, we identified the benign immune cell clusters as B-cells, NK cells, macrophages/monocytes, dendritic cells and various T-cell subtypes. For the non-immune cells in the TIME, fibroblasts were identified by COL1A2 and endothelial cells by VWF expression.

(5) tCTCL Exploits OXPHOS Metabolic Reprogramming, Cellular Plasticity and MYC/E2F Activities at Transformation

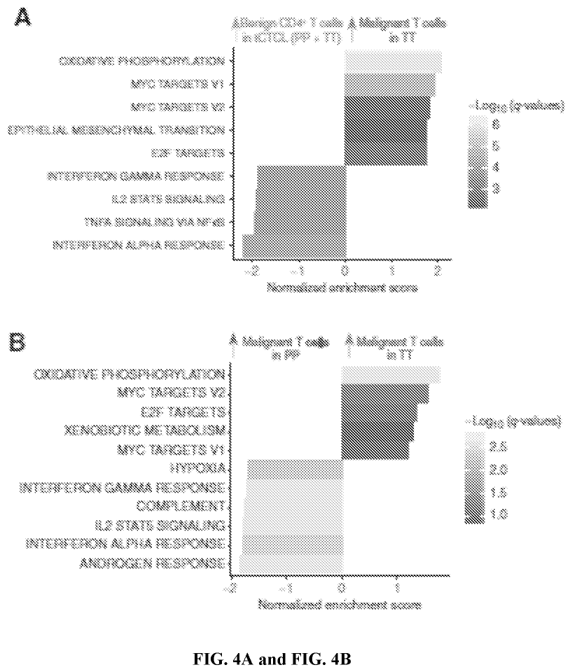

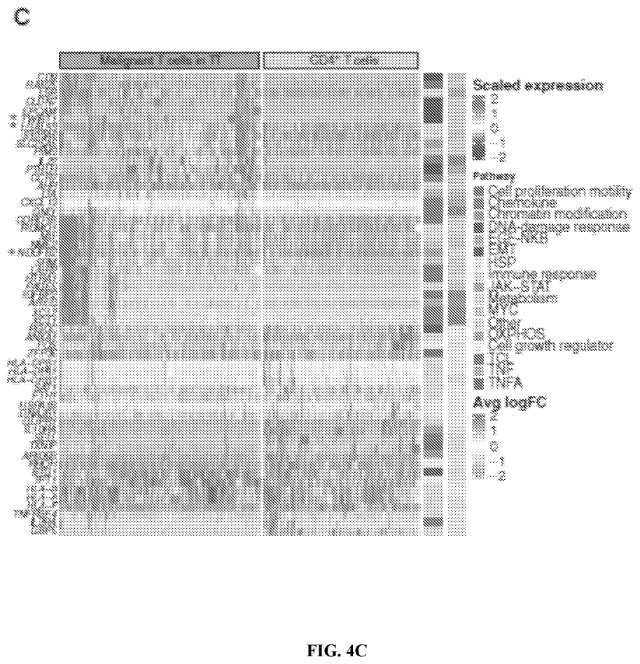

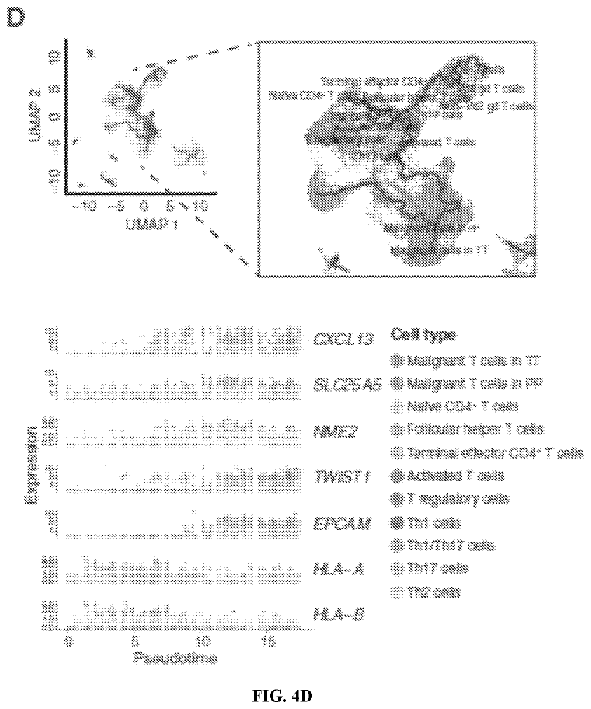

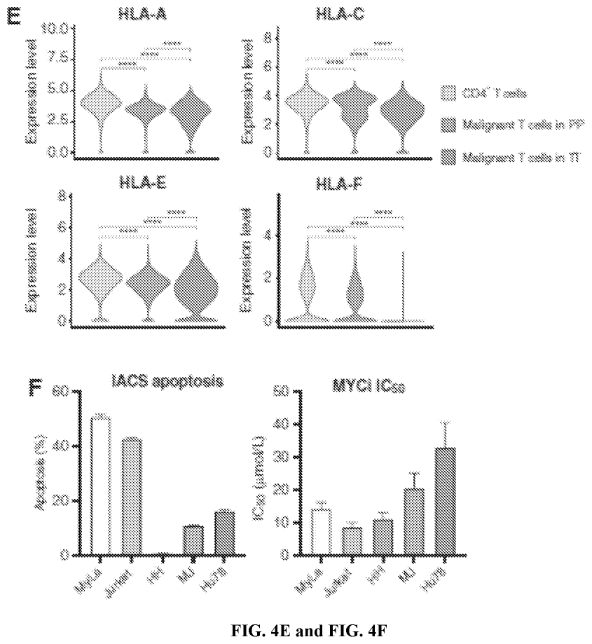

To define a malignant T-cell oncogenic program in tCTCL, we first performed differentially expressed gene (DEG) and gene set enrichment pathway analyses comparing malignant T-cells in TT and benign CD4+ T-cells in the TIME (FIG. 4A). Computational overlap of genes ranked by expression fold-change with the Molecular Signatures Database hallmark gene sets (MSigDB) showed significant enrichment of genes in OXPHOS, MYC, EMT and E2F target pathways and down-regulation of IFN-α, TNF-α, and IFN-γ (FIG. 4A). Importantly, significant upregulation of OXPHOS, MYC, and E2F target pathways and down-regulation of IFN-α, TNF-α, and IFN-γ pathways were also observed in disease evolution from PP to TT (FIG. 4B). Further analysis of the DEGs between malignant T-cells in TT vs benign CD4+ T-cells highlight a 55-gene malignant T-cell signature with upregulation of genes involved in oxidative phosphorylation (SLC25A5, NDUFB2), EMT/cellular plasticity and stemness (TWIST1, EPCAM, CLDN7), MYC signaling (NME2, SRM, PIM3), chemokines (CCR7), cell migration and motility (CD9/tetraspanin, LAIR2, RAB25), as well as down-regulation of HLA-A, HLA-B and HLA-F (MHC-I, IFN-γ pathway) that is indicative of immune escape in tCTCL (FIG. 4C). Of the upregulated genes, TWIST1 (cellular plasticity/stemness), NDUFB2 (OXPHOS) and LGALS3 (Galectin 3/RAS) are also predicted drivers within the GISTIC2.0 amplification loci and are therefore considered high confidence drivers in tCTCL.

We next examined the benign immune cell types in the tCTCL TIME. While there was no quantitative difference in benign CD4+ T-cells, CD8+ T-cells, macrophages, NK-cells and dendritic cells between concurrent PP and TTs, B-cells were significantly enriched in TTs and can play a pro-tumorigenic role during disease evolution.

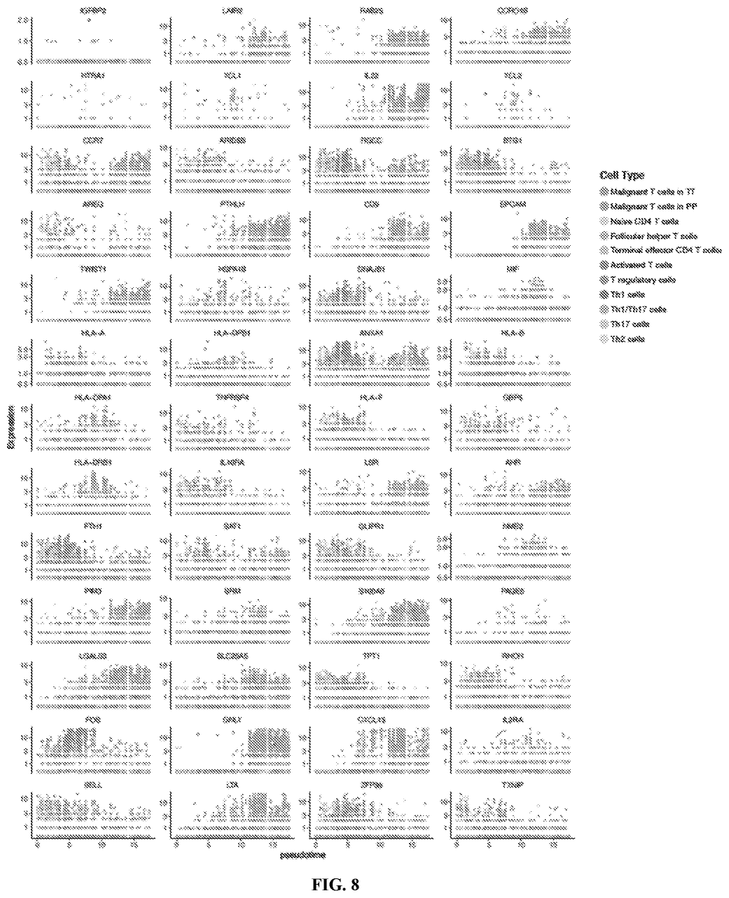

While scRNAseq data offers a high dimensional view of cell types, cell states and malignancy at single-cell resolution, these measurements ultimately represent a snapshot in time of a complex ecosystem with heterogeneous cell substrates in a diverse range of evolutionary stages. We therefore sought to order the trajectory of tCTCL constituents in pseudotime using Monocle 3, a machine learning algorithm that constructs the trajectory of cells between one of several possible end states along geodestic distance from a root node and can learn trajectories that have loops or points of convergence. We first arbitrarily assigned benign T-cells as the starting root node, here CD4+ naïve T-cells, and demonstrated a mono-directional trajectory from benign to malignant CD4+ T-cells in PP and TTs (FIG. 4D top). We next tracked the kinetics of the 55-gene malignant T-cell program along pseudotime and observed coordinated upregulation of CXCL13 (chemokine), SLC25A5 (OXPHOS) and NME2 (MYC) from benign to malignant PP and accentuation of cellular plasticity genes such as EPCAM, TWIST1 and PTHLH at the end of the trajectory in TT (FIG. 4D bottom; FIG. 8). Notably, downregulation of MHC-I genes (HLA-A, HLA-B and HLA-F) occurred early at the bifurcation from benign to malignant T-cells in PP (FIG. 4D), indicating immune evasion as an early event in CTCL lymphomagenesis, and is exacerbated from benign CD4 to malignant T-cells in PP to malignant T-cells in TT (FIG. 4E).

### (6) Small Molecule Inhibitors of OXPHOS and MYC Suppress CTCL Cells In Vitro

Our scRNAseq analyses identified OXPHOS and MYC as the most enriched pathways in tCTCL and therapeutic vulnerabilities. High oxidative phosphorylation is a hallmark of multiple hematopoietic malignancies and solid tumors. In pre-clinical models of acute myeloid leukemia and mantle cell lymphoma, inhibition of OXPHOS by IACS-010759, a potent small-molecule inhibitor of mitochondrial electron transport chain complex I, results in inhibition of tumor cell proliferation and induction of apoptosis. Likewise, MYCi975, a small molecule MYC inhibitor, is shown to have potent preclinical in-vivo activity against MYC. To determine the sensitivity of CTCL and other T cell lymphomas to OXPHOS and MYC inhibition, we performed pharmacologic perturbation assays using IACS10759 and MYCi975 in MF and other TCL cell lines (FIG. 4F). Using a group of established T-cell lymphoma lines, including Myla (MF), HH (MF—leukemic phase), and Hu78 (SS), we found low nanomolar sensitivity to IACS-010759 as assessed by apoptosis, particularly for Myla, and low micromolar sensitivity to MYCi975, as assessed by cell proliferation, cell viability and cytotoxicity (MTS assay). Together, these findings provide functional support for the two most significantly enriched malignant T-cell programs in tCTCL and use of OXPHOS and MYC inhibitors in treating aggressive T-cell lymphomas.

### (7) Analysis of Cellular Crosstalk Between Malignant T-Cells and the TIME Highlights MIF-CD74 Interactions

To systematically disentangle the complex intercellular network within the tCTCL ecosystem, we next investigated our scRNAseq data for receptor-ligand interactions between malignant T-cells and the TIME. We integrated our scRNAseq data with CellPhoneDB, a computational tool that interrogates scRNAseq data for protein subunit structures and receptor-ligand interactions, and explored the crosstalk between malignant T-cells and the macrophages/monocytes, B-cells, dendritic cells, NK cells, fibroblasts and endothelial cells (FIG. 5A). We showed top-ranking receptor-ligand pairs between MIF (ligand) in malignant T-cells and CD74, also known as HLA-DR antigens-associated invariant chain, in macrophages/monocytes, B-cells and dendritic cells (FIG. 5A). Importantly, our scRNAseq 55-gene malignant T-cell program also highlighted MIF as one of the significant differentially upregulated genes in malignant T-cells (FIG. 4C).

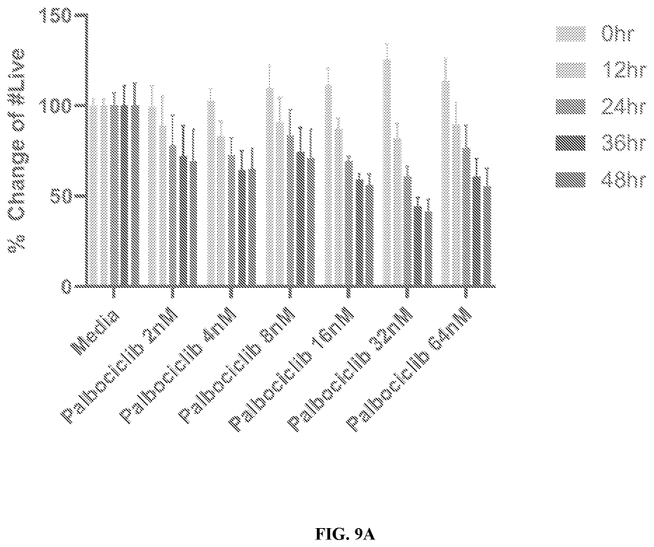

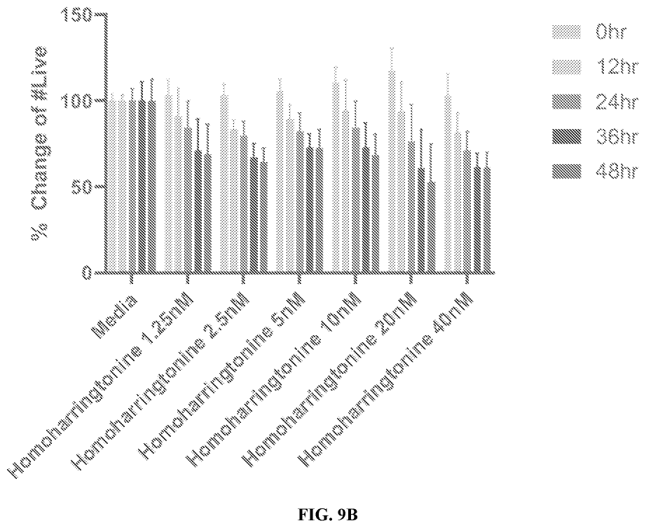

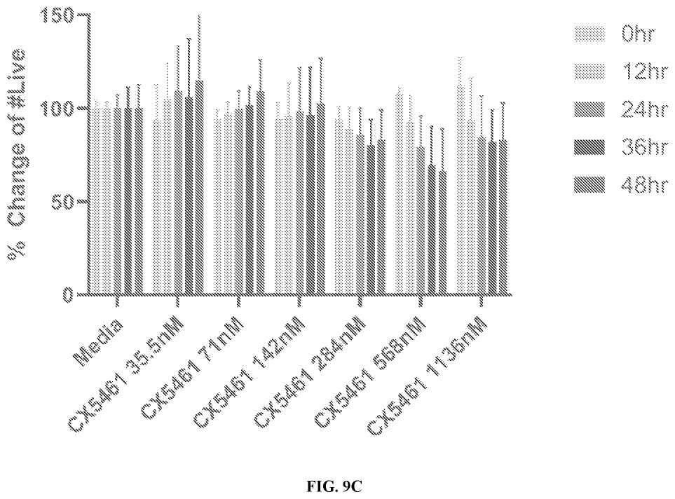

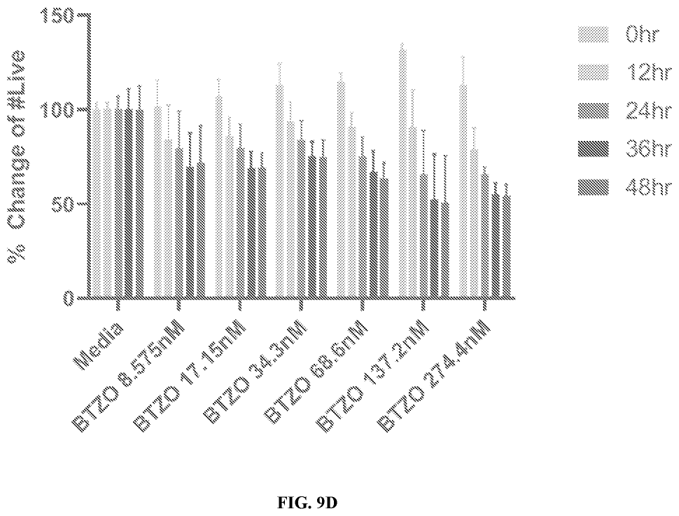

To demonstrate in situ evidence for MIF-CD74 interactions, we built a tissue microarray (TMA, 80 tissue cores) using skin biopsies from 21 tCTCL patients (16 with matched PP-Tumor LCT; 64 cores; FIG. 1A) and 9 PP patients without any history of transformation (PP, 16 cores). We performed immune profiling by Vectra multiplex immunofluorescence (mIF), and regions of interest (ROI) in each biopsy were selected from all lymphoid-dense areas in the biopsy. We stained this TMA with 2 panels of antibodies (anti-MIF, CD74, CD3, CD8, Ki67, CD68 antibodies and DAPI; anti-MIF, CD74, CD3, CD8, Ki67, PAX5 antibodies and DAPI) and imaged on Vectra 3. We analyzed a total of 488,399 cells by HALO software (Indica Labs) and demonstrated colocalization between MIF in malignant CD4+ T-cells (CD3+ CD8− Ki67+ cells) and CD74 in macrophages (CD68+) (FIG. 5B) and B-cells (PAX5+) (FIG. 5C), providing in situ validation for the CellPhoneDB predicted receptor-ligand interactions. These findings together indicate that MIF is an important therapeutic target in tCTCL through inhibiting MIF-CD74 interactions between malignant T-cells and APCs. Consistent with our observation in scRNAseq dataset, we also found moderate to prominent B-cell infiltrate in transformed tumors, while the PP lesions showed either minimal or no B-cell infiltrate (FIG. 5D). This TMA was also evaluated for differences in the TIME by CD30 expression (<:1%, >1% to <10%, 10-50%, >50%) via immunohistochemical studies. Here, we did not detect quantitative differences in macrophage, B-cell and CD8+ T-cell infiltrates or MIF-CD74 interactions between malignant T-cells and macrophages or B-cells.

(8) Dominant Subclones in tCTCL Show Upregulation of Genes Encoding Ribosomal Protein Subunits

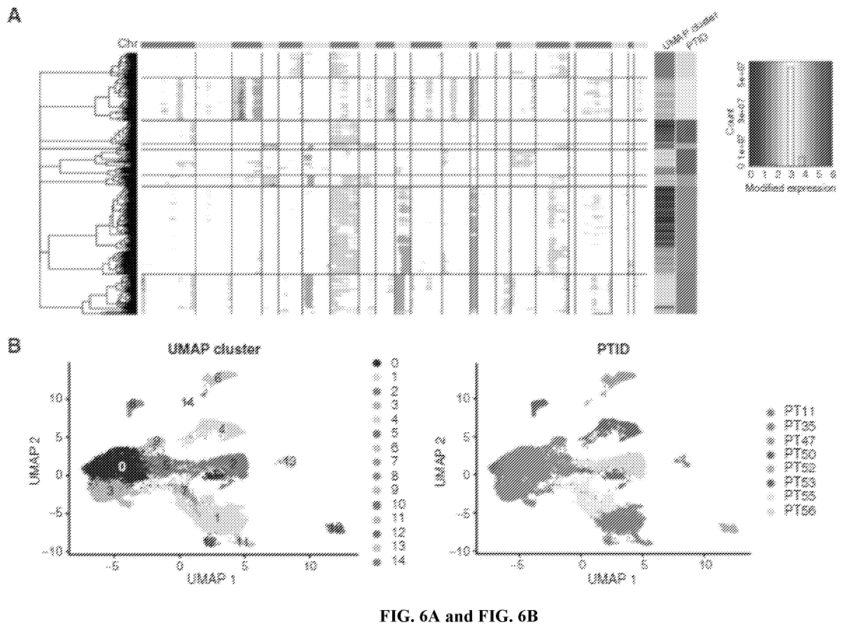

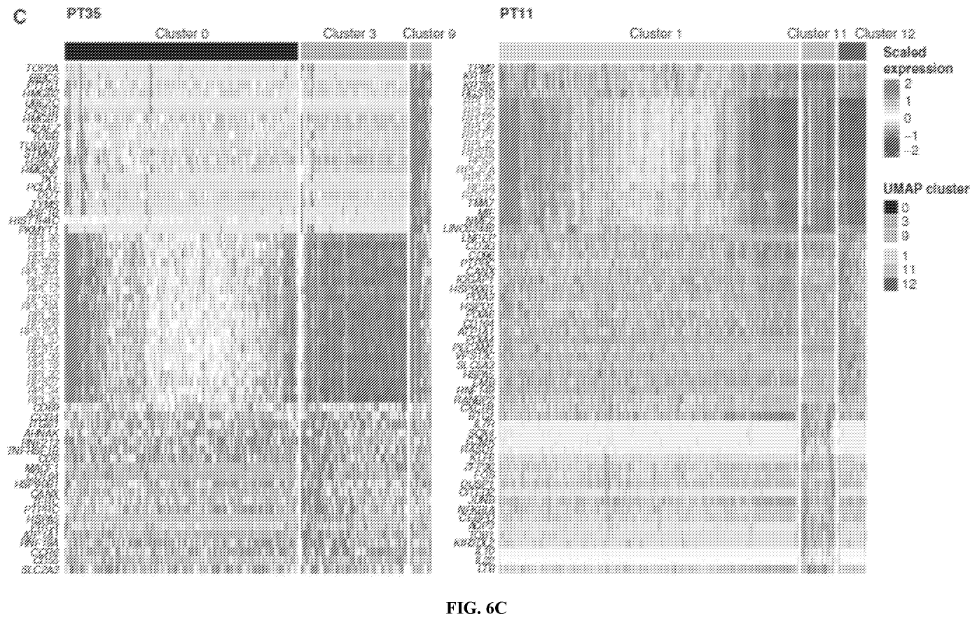

We have observed inter-patient tumoral heterogeneity in tCTCL (FIG. 3C), with some patients revealing more than one malignant T-cell clusters. We therefore sought to interrogate intra-tumoral heterogeneity (ITH) at the genetic and transcriptional level and determine if ITH correlates with patient outcomes. As CNV inference has been used to distinguish genetically distinct tumor subclones, we focused our attention on malignant T-cells from the scRNAseq dataset and performed CNV inference at the level of tumor subclusters using the inferCNV “subclusters mode” (qnorm method, FIG. 6A). We identified genetically distinct subclones of malignant T-cells within TTs from patients 11, 35 and 50 despite TCR monoclonality. Importantly, of the 8 patients in the scRNAseq cohort, PT 11 and 35 showed highest tumor burden (PT 11 TT clinical image FIG. 1A) and the worst clinical outcomes. UMAP clustering further demonstrated subclonal transcriptional heterogeneity in TTs from the same patients (PT11 cluster 1, 11, 12; PT 35 clusters 0, 3, 9; PT50 clusters 4, 8; FIG. 6B-C), with dramatically elevated ribosomal gene expression (large and small ribosomal protein subunits) in the preponderant malignant T-cell subclones (PT 11 and PT35). A similar ribosomal signature has been observed in circulating tumor cells from highly aggressive breast cancer. Our findings indicate therapeutic utility in targeting the ribosomal synthesis and global eukaryotic translational machineries for patients with aggressive, transformed CTCL.

(9) Cutaneous tCTCL and SS have Distinct Oncogenic Malignant T-Cell Programs

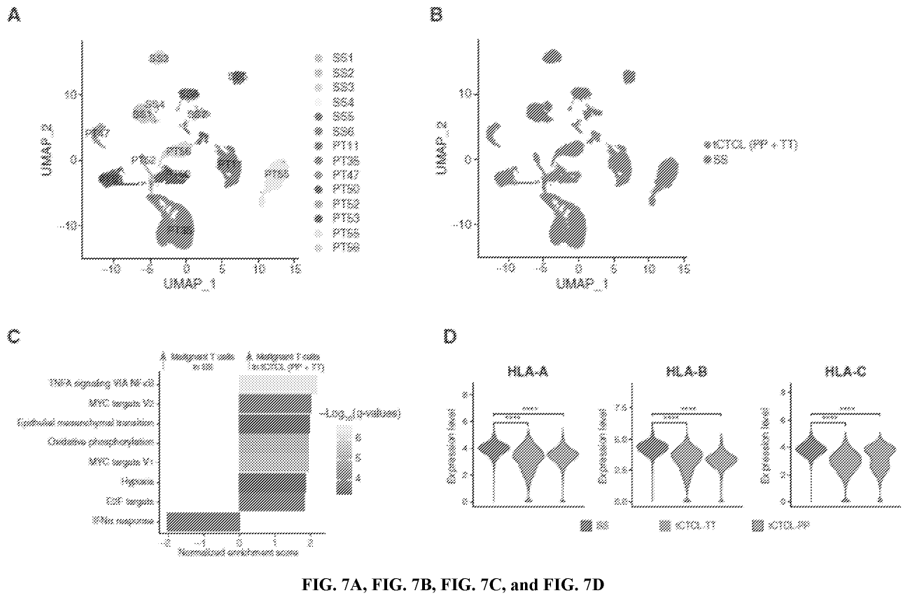

Finally, as we observed distinct patterns of CNA in tCTCL and SS (FIG. 2), we sought to investigate the differences in T-cell oncogenic signatures between these two advanced stages of CTCL at single-cell resolution. A recently reported SS cohort with matched single-cell transcriptomic (expanded CRISPR-compatible cellular indexing of transcriptomes and epitopes by sequencing, ECCITE-seq) and single-cell TCR clonotype (Herrera cohort) was downloaded for this analysis. Malignant T-cells from this SS cohort were identified by the dominant TCR clonotype and benign CD4+ T-cells by CD4+ polyclonal T-cells, and UMAP clustering showed clear separation between malignant T-cells versus benign CD4 T-cells. Interestingly, malignant T-cells in SS do not show upregulation of OXYPHOS and MYC activities or down-regulation of MHC-I genes compared to benign CD4 T-cells. We next compared the gene expression profile between malignant T-cells in tCTCL (PP and TT) to malignant T-cells in SS. Here, the malignant T-cells cluster by patients of origin, with tCTCL patient clusters clearly separating from the SS patient clusters (FIG. 7A), and with benign T-cells across different patients clustering together. Importantly, malignant T-cells in tCTCL (PP and TT) show significant upregulation of OXPHOS, MYC, EMT/stemness, E2F target and TNFα activities compared to malignant T-cells in SS, as well as down-regulation of IFNα pathway genes (FIG. 7C). Violin plots of MHC-I gene expression in malignant T-cells further revealed significantly lower HLA-A, B, C expression in tCTCL-TT and PP compared to SS (FIG. 7D), indicating immunosurveillance escape through loss of MHC-I as a more prominent feature in tCTCL.

To see the effect of various inhibitors on malignant T cells, we an EVOS drug assay (FIG. 9) measuring varying concentrations and time points for Palbociclib (CDK4/6 inhibitor)(FIG. 9A), Omacetaxine mepesuccinate (HHT)(FIG. 9B), CX5461 (rRNA synthesis inhibitor)(FIG. 9C), and BTZO-1 (MIF inhibitor)(FIG. 9D). By 12 hours, killing of malignant T cells was observed reaching a maximum for all drugs by 36 hours. BTZO-1, HHT, and Palbociclib treated cells showed that only 50% of target cells remained viable after treatment. CX5461 also showed killing to a lesser degree with closer to 60-70% target cells remaining viable after treatment.

### b) Discussion

Transformed CTCL is an aggressive large cell lymphoma that is resistant to multiple forms of systemic therapy. There is a critical unmet need for identifying novel therapeutic targets for this incurable cancer, yet its disease biology is poorly understood due to the multitude of challenges associated with tissue-based research in rare cancers. Genomic- and transcriptomic-level information of CTCL at disease transformation is limited, and a better understanding of tCTCL disease biology has broad implications for therapy. Our study contains the largest collection of tCTCL clinical specimens that are difficult to obtain and provides an important resource for the study of tCTCL biology and the identification of novel therapeutic vulnerabilities. We generated a WES dataset of similar size to other TCGA rare cancers, the first scRNAseq atlas of tCTCL, and immune profiled the TIME by scV(D)J seq and multiplex IF using serial biopsies from tCTCL patients. Together, this multi-omics study uncovered key driver events, prognostic mutational signatures, diverse oncogenic programs and receptor-ligand interactions that malignant T-cells exploit at large cell transformation.

Defining the T-cell lymphoma ecosystem at single-cell resolution is particularly challenging as the TIME is comprised of malignant CD4+ T-cells, benign T-cells, as well as other diverse immune cells. Gene expression-based clustering is imprecise in defining malignancy, and dropout read is a well-known phenomenon in scRNAseq and scVDJseq datasets. Here, we implemented a robust two-tiered algorithm using simultaneous single-cell whole transcriptome-V(D)J profiling and chromosomal copy number inference to identify malignant T-cells from the TIME, and we believe this methodology can be applied to the study of other T- and B-cell leukemia/lymphomas. While CTCL is clinically viewed as a cancer of monoclonal T-cells, a recent study showed TCR clonotypic diversity in CTCL by bulk TCR seq and indicated that T-cell tumorigenesis occurs prior to TCR gene rearrangement. In tCTCL, we observed TCR monoclonality, with monoclonal T-cells showing a malignant CNV patterns, and background polyclonal T-cells in the TIME showing neutral CNV patterns. The difference in these two studies could be due to differential resolution of TCR detection using scV(D)Jseq, with the ability to assign precise TCR α- and β-sequence pairs to individual T-cells. Nevertheless, our data indicate that TCR monoclonality is a key feature of transformation. Future single cell TCR studies involving CTCL lesions at different stages of disease can elucidate the origin of CTCL with respect to the timing of TCR gene rearrangement.

A central finding from our single-cell study is the diverse oncogenic programs that malignant T-cells exploit to confer aggressive behavior and survival advantage at transformation, with upregulation of OXPHOS and MYC as the top enriched pathways, which are progressively upregulated in disease evolution, followed by EMT/stemness and E2F target genes (FIGS. 4A and 4B). Notably, similar OXPHOS and MYC upregulation was not observed in our single-cell analysis of an independent SS cohort (FIG. 7C,). Metabolic reprogramming toward oxidative phosphorylation, and hence increased ATP production, has been reported in other high grade leukemias, lymphomas and solid tumors. In tCTCL, this metabolic shift can be viewed as advantageous for supplying the energy demand of rapidly growing, high-grade lymphoma cells. As MYC activation has been shown to drive cancer cell metabolism toward glutaminolysis and mitochondrial biogenesis, the shift toward OXPHOS can be related to MYC upregulation. Interestingly, quiescent T-cells (naive or memory T-cells) are known to use catabolic metabolism by oxidative phosphorylation to fuel cell survival, raising the possibility that the upregulation of OXPHOS in tCTCL can also be related to T-cell state at transformation. While future studies are needed to chart the mechanistic action of these pathway components, it is encouraging that novel small molecule inhibitors of OXPHOS (IACS-10759) and MYC (MYCi975) provided functional support of OXPHOS and MYC as therapeutic vulnerabilities. In addition to OXPHOS and MYC, multiplex IF studies demonstrated interactions between malignant T-cells expressing MIF and macrophages and B-cells expressing CD74 in situ.

A recurrent theme observed in this study is the up-regulation of genes involved in cellular plasticity and stemness in tCTCL. In our WES dataset, GISTIC analyses aimed at detecting genomic loci targeted by SCNAs revealed recurrent amplifications in TWIST1, a master transcription factor of EMT/stemness, while the scRNAseq 55-gene malignant T-cell signature demonstrated upregulation of TWIST1, CLND7 and EPCAM, all involved in cellular plasticity/stemness. We also observed recurrent mutations in Hippo pathway genes such FAT1 (˜30%), loss of which has been implicated in driving tumor cell stemness and metastasis in cutaneous squamous cell carcinoma. Tumor plasticity is a key feature of aggressive cancer transition, which can certainly account for the transition of CTCL to an aggressive large cell lymphoma with blast-like morphology at transformation. In large-scale genomics studies across 21 solid tumors, activation of stemness programs was shown to positively correlate with ITH and drives clonal evolution while limiting anti-tumor immune responses. Indeed, we observed ITH in tCTCL, with prominent subclonal upregulation of ribosomal subunit gene expression in patients with the worst clinical outcomes.

Besides the upregulated signatures, one intriguing observation from our single-cell dataset is the downregulation of MHC-I in malignant T-cells in tCTCL. Comparison of MHC-I gene expression levels (HLA-A, C, E, F) showed progressive and significant decreases from benign CD4 T-cells to PP to TT, and our pseudotime analysis corroborated these changes in expression across disease evolution. While high TMB cancers are theoretically more immunogenic and responsive to T-cell based immunotherapies, loss of MHC-I antigen presentation can compromise the visibility of tumor cell antigens to CD8+ T-cells and response to checkpoint blockade agents. In such MHC-I negative or low tumors with defective antigen presentation machinery, NK-cell based therapies and epigenetic drugs can be considered.

The role of B-cells in the tumor microenvironment is recently under intense investigation in various cancer types, though it is still incompletely explored compared to other benign immune substrates of the TIME. The results are also variable, with some studies showing positive correlation between B-cell infiltrate and patient outcomes or response, while other studies suggest a pro-tumorigenic role of B-cells. In tCTCL, our scRNAseq data shows significant enrichment of B-cell infiltrate mostly in transformed tumors, a finding that is supported by multiplex IF. Altogether, our results indicate a pro-tumorigenic role of B-cells in tCTCL, possibly through interactions with malignant T-cells via MIF-CD74 signaling.

Lastly, CTCL is a rare cancer with well-known racial disparity. Black/AA patients have a higher incidence rate, younger age of onset, higher disease burden and inferior survival compared to Whites, even after accounting for disease characteristics, socioeconomic factors, and treatments received. Nonetheless, biological factors underlying these racial disparities are poorly understood. Here, we provide the first attempt at identifying genomic correlates for the survival gap between Black/AA and White patients. While the sample size is admittedly small, we observed a significantly lower contribution of the UV signatures that are prognostic for favorable survival and enrichment of other signatures that can drive worse outcomes in the Black/AA patients.

Rare cancers represent one of the greatest inequalities in cancer research, with the lack of well-curated tissue specimens to study disease biology and resources for pharmaceutical development. Transformed CTCL exemplifies such challenge where the lack of genomic and transcriptomic level information contributes to the paucity of drug discovery efforts. In this study, we have identified genetic driver events and pathways in tCTCL and elucidated a malignant T-cell oncogenic program with novel therapeutic vulnerabilities. While further validation in larger cohorts and pre-clinical models are needed, our investigation provides a key resource with the largest collection of tCTCL samples studied to date.

### c) Methods

### (1) Data Process and Somatic Mutation Filtering

NGS data was processed with a well-established in-house bioinformatics pipeline (Sanghoon Lee, Li Zhao, Cell Report 2020). In brief, raw sequencing base call (BCL) files were first converted into FASTQ files with Illumina bcl2fastq2 conversion software v2.20. Then DNA sequences were aligned against the hg19 reference genome using the BWA software (v0.7.15, Li and Durbin, 2009). The Picard toolset was used to convert data into a BAM format and remove read duplicates (v1.112, http://broadinstitute.github.io/picard/). Finally, the GATK toolkit was used to perform local realignments.

We used MuTect and Pindel to identify somatic single nucleotide variants (SNVs) and small insertions and deletions (indels), respectively. Then, with ANNOVAR (Version 20190ct24), we annotated each genetic variant with coding sequence change and allele frequency in control populations, including Exome Aggregation Consortium (ExAC), Genome Aggregation Database (gnomAD), the 1000 Genome Project (, and NHLBI-ESP 6500 exomes. A set of filters were applied to select somatic mutations of a good sequencing quality: (1) total reads: ≥20 reads in the tumor sample and ≥10 reads in normal sample (matched germline sample or merged normal tissues); (2) variant allele frequency (VAF): for SNVs, VAF≥0.02 in tumor sample and ≤0.02 in normal sample; for indels, VAF in tumor sample ≥0.05 and no observation in normal tissue; (3) length of indels ≤100 bp; (4) excluding intronic and intergenic mutations; (5) population allele frequency <0.01 in all of the four control databases.

### (2) Cancer Driver Gene Detection

NGS data were processed using established in-house bioinformatics pipeline. To enhance the coverage of our analysis, two mutation-based computational algorithms were applied, MutSigCV (version 1.41) and dNdScv (RRID: SCR_017093). MutSigCV was run with default parameters using the MATLAB (RRID:SCR_001622) Compiler Runtime (MCR). A global q-value threshold of 0.1 was applied for selecting driver genes as indicated by the MutSigCV developers. dNdScv was run in R (version 3.6.3) using the default parameters. A global q-value (qallsubs_cv) of <0.05 was used as a cut-off for selecting predicted driver genes.

### (3) Copy Number Variantion Detection and Analysis

CNV analyses were performed in samples with matched germline. CNVs were identified using R package ExomeLyzer. Copy number change was determined as the log 2 ratio of tumor versus normal reads, and circular binary segmentation (CBS) algorithm was applied for CNV segmentation. To identify genomic regions significantly enriched with CNVs, GISTIC2.0 (RRID:SCR_000151) was used with default settings. Genomic regions with FDR <0.25 were considered as significantly enriched and highlighted in the chromosome landscape plot.

### (4) TMB

TMB analysis was performed in all samples with matched germline/normal tissue using Maftools (v2.6.0). Results were compared to 33 TCGA cancer types (MC3 data). TMB for samples that have a matched normal tissue sequenced was calculated by the following formula

$$T M B = \frac{\#non - synonymous mutations}{sequencing capture size ( Mb )}$$

where the sequencing capture size of the current study is 39 Mb and that of the TCGA studies is 35.8 Mb after data harmonization. Results were compared to 33 TCGA cancer types (MC3 data) and two SS cohorts (Wang cohort and Choi cohort). The Choi cohort raw sequencing data was downloaded and processed using the same computational pipeline as our tCTCL cohort. The Wang cohort TMB was calculated using processed mutations from the original publication. The sequencing capture size is 39 Mb for the study cohort and 35.8 Mb for the TCGA cancer cohorts after data harmonization.

### (5) Mutational Signature Analysis

We analyzed the mutational signatures at both a cohort- and a tissue-level. To decipher the dominant mutagenic process of the whole cohort, we extracted de novo representative signatures from mutation catalogs of the 70 CTCL-MF samples using Maftools. In details, a mutation context matrix was first built; then four signatures were determined as optimal in representing the mutation profile and further extracted with the non-negative matrix factorization (NMF) method; finally the constructed signatures were compared against pre-defined mutational signatures (COSMIC version 3 signatures).

To further compare the mutational signature pattern across different tissues, we estimated the contributions of known signatures (COSMIC version 3 signatures) in each tissue independently using two R packages, MutationalPatterns and deconstructSigs. The MutationalPatterns was used to calculate a cosine similarity between the mutational profile of each tissue and each of the COSMIC reference signatures, enabling a direct pairwise comparison. The deconstructSigs, on the other hand, was used to determine a combination of known signatures that can most accurately reconstruct the mutation profile of an individual tumor tissue. Such combination was quantified with the contribution weights of each reference signatures. Both tools were run with the default settings.

### (6) Survival Analysis

For Kaplan-Meier survival analysis, survival R package (version 3.2-7) was used. Survival analysis was performed from time of initial LCT to the date of death (event time) or the date of last follow up (censoring time). The patients were grouped into two sub-groups according to the median of genomic features, such as the weighted contribution of SBS7 signature. For patients with paired PP and TT tissues, data from the TT sample was used for survival analysis. Survival plot was generated using the survminer R package.

### (7) Analysis of Highly Mutated Pathways

A variety of cancer-associated pathways were assessed for a mutational enrichment in the CTCL-MF cohort: (1) eight oncogenic signaling pathways that have been summarized by the TCGA project and implemented in the Maftools; (2) four pathways compiled from review papaers, including cell cycle, TCF-beta signaling, MMR, and UV expose. Genes with non-synonymous mutations observed in ≥3 tissues were visualized as an oncoplot using Maftools.

### (8) Tissue Collection

Tumors were minced in small fragments on a Petri dish and dissociated using a solution of 2 mg/mL collagenase IV (160U/mg, Thermo Fisher Scientific, Waltham, MA), 0.1 mg/mL DNase I (Roche, Basel, Switzerland) and 0.1 mg/mL hyaluronidase (1000 U/mL, Worthington Biochemical, Lakewood, NJ) diluted in RPMI (Coming, New York, NY) supplemented with 10% fetal bovine serum (FBS) (Gemini Bio-Products, West Sacramento, CA). Tumors were incubated with the dissociation buffer for 1h at 37C at 5% CO₂. After digestion, samples were carefully homogenized with a 18G syringe, filtered through a 70 μM cell strainer and washed with RPMI supplemented with FBS. Supernatant was then removed, and samples were resuspended in PBS (Thermo Fisher Scientific). After quantification, cell concentration was adjusted following manufacturer's recommendation to sequence a target of 10,000 cells per sample.

### (9) Data Filtering, Normalization and Cell Clustering

The Seurat (v3 and v4) tool kit was used for data process. A Seurat object was first constructed with the Cell Ranger outputs. A series of filters were then applied for data quality control. Cells meeting the following criteria were remained for the analysis: (1) count of UMIs per cell ≥500; (2) number of detected genes per cell ≥500;

$${log}^{\frac{\# of detected genes}{\# of UMIs}} > 0.8 ; \qquad ( 3 )$$

(4) percentage of sequencing reads mapping to mitochondrial genes <0.2. In addition, genes expressed in less than 10 cells were excluded.

The after-filtering Seurat object was split into PP and tumor subsets given the cell origin. Each subset was normalized separately using the SCTransform method with regressing cell cycle scores and mitochondrial mapping percentage. Then the two subsets were merged with the Seurat integration pipeline, where 3,000 features were used to search integration anchors. The PCA analysis and the UMAP method were applied on the ‘integrated’ data assay to reduce data dimension. Cells with a similar expression pattern were placed nearby with a K-nearest neighbor (KNN) graph-based approach implemented in the FindNeighbors function, followed by clustering partitioning with the FindClusters function.

### (10) Identification of Malignant T Cells

The malignancy status of a high confidence was determined by analyzing the scV(D)J sequencing data. We first processed and filtered the sequencing data with the Cell Ranger pipeline and selected the contigs that were predicted as productive for the downstream analysis. Then in each scV(D)J profile, the dominant clonotype was determined with the most numerous clonotype and other clonotypes that share partial overlap in CDR3S amino acid sequence. For instance, the dominant clonotype in PT47 PP tissue is TRA:CALSEVNYGGSQGNLIF (SEQ ID NO: 1);TRB:CASSQSRTVYGYTF (SEQ ID NO: 2). Due to dropout events, cells with a clonotype of either TRA: CALSEVNYGGSQGNLIF (SEQ ID NO: 1)or TRB:CASSQSRTVYGYTF (SEQ ID NO: 2)were dominant clonal cells as well. Herein, ‘true malignant’ T cells were those of a dominant clonotype and ‘true benign’ T cells were defined with an observation of a non-dominant clonotype.

Since not all of the cells were captured by the scV(D)J-seq, we aimed to retrieve additional malignant T cells from cells without a valid clonotype. First, we identified additional T cells by selecting those expressing T cell marker genes (CD3E, CD3D, or CD3G) and named them as ‘malignant suspect’ cells. Second, we applied a clustering approach to determine whether a malignant suspect cell is more similar to ‘true malignant’ or ‘true benign’ cells. The clustering is based on the CNV pattern of each individual cell, since tumor genomes tend to feature an increased number of CNVs and a complex rearrangement pattern. The R package inferCNV (version 1.3.5) was used to extract CNV patterns and perform the clustering. In details, inferCNV employs a reference cell set (benign cells of the same cell type) to de-noise any background CNV signal and identify CNVs on the observation cells. Herein, we split the ‘true benign’ cells into reference cells for de-noising control and observation cells for cell clustering. To ensure a similar ratio of CD4⁺ (expressing CD4) and CD8⁺ (expressing CD8A or CD8B) cells in both cell sets, one third of each type were randomly selected as observation cells and the remaining ‘true benign’ cells were grouped as reference cells. InferCNV was run with enabling the ‘denoise’ mood but turning off the ‘cluster by_groups’ parameter to cluster observation cells regardless of tissue origin. The dendrogram was cut into 16 subclusters. As last, the ‘malignant suspect’ cells in each subcluster were further assessed as either malignant or benign given the CNV patterns and the composition of ‘true malignant’ and ‘true benign cells’.

### (11) Cell Type Annotation of Benign Cells

Benign immune cells were determined from the non-malignant T cells with either a non-dominant clonotype or an expression of PTPRC, a cell marker for CD45⁺ immune cells. We identified the cell types by cross referencing two analyses: manual analysis of marker genes with prior knowledge and an automatic annotation approach using the SingleR R package. The benign immune cells were clustered (see Methods—data normalization and cell clustering), followed by extraction of marker genes using the FindAllMarkers function of Seurat. With known knowledge of marker genes, cell clusters were manually annotated as T cells, B cells, dendritic cells, macrophage/monocytes, and NK cells. To further refine annotations for benign T cells, we performed a second clustering on T cells only and identified cell clusters of activated T cells, T regulatory cells, exhausted CD8⁺ T cells, and Thl7 cells. Next, we used SingleR to determine a cell type for the remaining T cells which lack adequate knowledge on the marker genes. SingleR assesses each scRNA-seq profile independently by correlating it with reference transcriptomes of pre-defined cells. To run SingleR, we first constructed a raw count matrix of the scRNA-seq data in a SingleCellExperiment object and then normalized it with a log-transformation using scater R package. The reference transcriptomes in use were from the Monaco immune datasets (GSE107011), which summarized bulk RNA sequencing profiles of sorted immune cell lines, covering comprehensive immune cell types. The SingleR results were further validated by cross-referencing cell types defined with marker gene expression and used to determine a cell type for the remaining T cells. At last, out of the non-immune cells, we identified fibroblast and endothelial cells with marker gene expression (COL1A2 and VWF, respectively).

### (12) Differential Expression Analysis

The differential expression analysis was performed using the Wilcoxon rank sum method with two Seurat functions: FindMakers for pairwise comparisons and FindAllMarkers for comparisons across multiple groups. Both functions were run on the ‘RNA’ data assay with no pre-filters. Significant differential expression genes (DEGs) were filtered with a q value <0.05 and an absolute value of fold change (FC) >2. To reveal pathways in which DEGs are enriched, a GSEA was tested on all of the detected genes ranked by average logFC. The analysis was performed with the clusterProfiler R package against pre-defined hallmark gene sets of the MSigDB database.

### (13) Trajectory Analysis

T cells, both malignant and benign, were first constructed in a Seurat object and processed and clustered as described above. The trajectory analysis was performed using the Monocle 3 pipeline. To meet the format requirement of Monocle 3, the Seurat object along with the clustering information was converted as a cell data set using SeuratWrappers R package. Trajectory was then learned with the default parameters of Monocle 3 leam_graph function. Assuming that cells differentiate from the root of the trajectory to the end leaves, we manually selected naïve CD4⁺ T cells in the graphical interface as the beginning point and ordered cells in pseudotime. Next, a branch of the trajectory graph originating from naïve CD4+ T cells to malignant T cells was further subset using the choose_graph_segments function. The expression change of selected genes was plotted along pseudotime of the selected branch cells. To construct patient-specific trajectories, we applied the same approach for cell clusters of malignant T cells from each patient and combined benign T cells.

### (14) Intratumor Heterogeneity Qualification

To reveal the degree of intratumor heterogeneity for each patient, we clustered malignant T cells using both RNA expression data and CNV patterns. First, malignant T cells of eight patients were first processed and clustered according to gene expression features as described above. The ITH degree can be referred from the cluster numbers of each patient. Second, inferCNV was performed on malignant T cells using benign T cells as reference. This time, the program was run with restricting patient-specific clustering by setting the parameter cluster_by_groups as True. In addition, the analysis mode of ‘subclusters’ was enabled with the qnorm partition method to reveal ITH patterns. Furthermore, cells were clustered based on similarity of the extracted CNV patterns. At last, we compared the two clusterings by plotting the expression clusters next to the inferCNV output using the same cell order.

### (15) Cellular Communication Analysis

CellPhoneDB was used to infer top ligand-receptor pairs for each pair of the previously determined cell types, including malignant T cells, benign immune cells, fibroblasts, and endothelial cells. The raw count matrix of single-cell expression profiles was normalized as introduced in the CellPhoneDB paper (CellPhoneDB: Inferring cell-cell communication from combined expression of multi-subunit ligand-receptor complexes) where the count value of each gene each cell was divided by the total counts of corresponding cell, followed by multiplying 10000. CellPhoneDB was run with the ‘statistical’ method in the virtualenv environment. The parameters were set as default. Ligand-receptor pairs of a p value <0.05 were filtered as significantly enriched pair. To visualize the results, we selected the top 3 ligand-receptor pairs in log 2mean expression value for each cell type pairs and summarized the results with a dot plot using R package ggplot2.

### (16) Patient Cohort and Clinical Annotation

Skin biopsies from 56 patients with tCTCL were collected and analyzed under Institutional Review Board (IRB) approved protocols (MCC19672, MCC14690) with appropriate written informed consent. The studies were conducted in accordance with the Declaration of Helsinki and approved by the Moffitt Cancer Center IRB. Electronic medical charts were reviewed independently by two investigators to document the clinical parameters, including age, gender, time of first rash, time of initial MF diagnosis, time of LCT, time of death/last follow up, clinical staging at time of initial MF diagnosis or presentation to MCC, clinical re-staging at time of LCT, and treatment history.

### (17) Clinical Tissue Samples

Of the 56 tCTCL patients, 54 had available tissues for WES (n=70 skin formalin-fixed, paraffin-embedded (FFPE) biopsies) (FIG. 1A). All samples were assessed by two pathologists for adequacy of lymphoid infiltrate, including 45 TT and 25 PP lesions (9 with LCT). 17 patients had paired PP and TT (11 precursor PP, 6 concurrent PP to the LCT lesion). Matched germline samples were available from 41 patients, while remaining patient samples used panel of normal as control. Tumor samples were microdissected before submitted for WES.

### (18) Patient Sample Collection

Fresh tissue specimens were collected from 8 patients with tCTCL treated and managed at the H. Lee Moffitt Cancer Center Cutaneous Oncology and Malignant Hematology clinics. Two samples were collected per patient (concurrent PP and TT lesions). Informed consent and fresh tissue biopsies were collected under institutional Total Cancer Care IRB protocol (MCC 14690, main and research biopsy addendum) and processed under MCC19672.

### (19) Single-Cell V(D)J-Seq for TCR Clonotype

TCR annotation was performed using Cell Ranger “vdj” function for sequence assembly and paired clonotype calling. Paired TCR α/β reads attributed to each cell barcode were grouped and assembled into a single contig to determine the combined TCR α/β clonotype, and contigs that were predicted as productive were selected for the downstream analyses.

### (20) Single-Cell CNV Inference

124. InferCNV v1.3.5 was used to extract chromosomal CNV patterns. Two-thirds of non-dominant TCR clonotype T-cells (CD4+, CD8+) were used as reference normal cell group for de-noise control. For the observation cell group, 1) the remaining non-dominant TCR clonotype cells, 2) all dominant TCR clonotype (“true malignant”) cells and 3) all CD3+ T-cells with TCR dropout (“malignant suspect”) were included. InferCNV was run using “denoise” mode, and “cluster by group” parameter was turned off such that observation cells can cluster regardless of tissue of origin.

### (21) Analysis of Intra-Tumoral Heterogeneity

InferCVN subcluster analysis for ITH at the genetic-level was performed on malignant T-cells (identified based on scV(D)J seq and copy number inference method as described above). Patient-specific clustering was restricted by setting the parameter “cluster by_groups” as “True”. ‘Subclusters’ mode was enabled with the qnorm partition method to reveal ITH patterns. Malignant cells were clustered based on similarity of the extracted CNV patterns. Gene expression-based clustering of malignant T-cells by UMAP was performed to evaluate for ITH at the transcriptional-level.

### (22) Independent SS Cohort Analysis (Herrera Cohort)

To perform single-cell transcriptomic analysis of SS and malignant T-cell comparison between tCTCL and SS, ECCITE-seq scRNAseq gene expression counts and TCR α/β clonotype profiles from 6 SS blood samples (SS1-SS6) were downloaded from the NCBI GEO database (GSE171811).

### (23) Pharmacologic Perturbation Assays

Established T-cell lymphoma lines were used for drug assays, including Myla (MF; Sigma Aldrich Cat #95051032), HH (MF—leukemic phase; ATCC Cat #CRL-2105), and Hu78 (SS; ATCC Cat #TIB-161), Jurkat (ATLL) and MJ (ATLL; ATCC Cat #8294). For the apoptosis assay, indicated cell lines were seeded with the density of 1×10⁵ cells/well in 96-well plate and treated with 8 nM of IACS-010759 (SelleckChem #S8731). At day 5, cells were harvested and stained with Annexin V and PI following manufacture's protocol (Biolegend Cat#640914). Annexin V⁺PI⁺ population was gated on Singlet population using FlowJo 10 software (RRID:SCR_008520). For cell proliferation assay, indicated cell lines were seeded with the density of 1×10⁴ cells/well in 96-well plate and treated with different dose of MYCi975 (MedChemExpress Cat #HY-129601) for 5 days. At day 5, cells were incubated with MTS reagent following manufacture's protocol (Promega Cat #G3580). Absorbance at OD490 nm was recorded and percentage of growth were normalized to vehicle control. IC50 was calculated based on curve fitting result using non-linear regression function of GraphPad Prism 8 (RRID:SCR_002798).

### (24) Vectra Multiplex Immunofluorescence

An 80-core tissue microarray (TMA) was built using skin biopsies from 21 tCTCL patients (64 tissue cores, including 16 patients with matched PP and TT) and additional 9 patients with PP lesions and no history of LCT (16 tissue cores). ROIs were selected based on the most lymphoid-dense/representative area in the tissue. Quantitative Image Analysis was performed using the HALO Image Analysis Platform (Indica Labs, Albuquerque, NM).

### (25) Statistical Analysis

Statistical analysis was performed using R programming language (version >3.6). In survival test, a two-tailed log-rank test was used to determine the difference between patient subgroups. The statistical significance between category groups was determined using Wilcoxon rank sum test. ns, p >0.05; *, p<0.05; **, p<0.01; ***, p<0.001; ****, p<0.0001. For comparison of survival between patient groups (FIG. 1D,), data from the TT sample was used when a patient had paired PP and TT available.

### D. REFERENCES

Alexandrov L B, Kim J, Haradhvala N J, Huang M N, Tian Ng A W, Wu Y, et al. The repertoire of mutational signatures inhuman cancer. Nature 2020; 578(7793):94-101 doi 10.1038/s41586-020-1943-3.
Angelo M, Bendall S C, Finck R, Hale M B, Hitzman C, Borowsky A D, Levenson R M, Lowe J B, Liu SD, Zhao S, Natkunam Y, Nolan GP. Multiplexed ion beam imaging of human breast tumors. Nat Med. 2014 April; 20(4):436-42.
Arulogun S O, Prince H M, Ng J, Lade S, Ryan G F, Blewitt O, et al. Long-term outcomes of patients with advanced-stage cutaneous T-cell lymphoma and large cell transformation. Blood 2008; 112(8):3082-7 doi 10.1182/blood-2008-05-154609.
Ashton T M, McKenna W G, Kunz-Schughart L A, Higgins G S. Oxidative Phosphorylation as an Emerging Target in Cancer Therapy. Clin Cancer Res 2018; 24(11):2482-90 doi 10.1158/1078-0432.CCR-17-3070.
Blokzijl F, Janssen R, van Boxtel R, Cuppen E. MutationalPatterns: comprehensive genome-wide analysis of mutational processes. Genome Med 2018; 10(1):33 doi 10.1186/s13073-018-0539-0.
Borcherding N, Voigt A P, Liu V, Link B K, Zhang W, Jabbari A. Single-Cell Profiling of Cutaneous T-Cell Lymphoma Reveals Underlying Heterogeneity Associated with Disease Progression. Clin Cancer Res. 2019; 25(10):2996-3005.
Bradford P T, Devesa S S, Anderson W F, Toro J R. Cutaneous lymphoma incidence patterns in the United States: a population-based study of 3884 cases. Blood. 2009; 113(21):5064-5073.
Campbell B B, Light N, Fabrizio D, Zatzman M, Fuligni F, de Borja R, et al. Comprehensive Analysis of Hypermutation in Human Cancer. Cell 2017; 171(5):1042-56 e10 doi 10.1016/j.cell.2017.09.048.
Cao J, Spielmann M, Qiu X, Huang X, Ibrahim D M, Hill A J, et al. The single-cell transcriptional landscape of mammalian organogenesis. Nature 2019; 566(7745):496-502 doi 10.1038/s41586-019-0969-x.
Caro P, Kishan A U, Norberg E, Stanley I A, Chapuy B, Ficarro S B, et al. Metabolic signatures uncover distinct targets in molecular subsets of diffuse large B cell lymphoma. Cancer Cell 2012; 22(4):547-60 doi 10.1016/j.ccr.2012.08.014.
Choi J, Goh G, Walradt T, et al. Genomic landscape of cutaneous T cell lymphoma. Nat Genet. 2015; 47(9):1011-1019.
da Silva Almeida A C, Abate F, Khiabanian H, et al. The mutational landscape of cutaneous T cell lymphoma and Sezary syndrome. Nat Genet. 2015; 47(12):1465-1470.
Dang C V. MYC, metabolism, cell growth, and tumorigenesis. Cold Spring Harb Perspect Med 2013; 3(8) doi 10.1101/cshperspect.a014217.
DeSantis C E, Kramer J L, Jemal A. The burden of rare cancers in the United States. CA Cancer J Clin 2017; 67(4):261-72 doi 10.3322/caac.21400.
Diamandidou E, Colome-Grimmer M, Fayad L, Duvic M, Kurzrock R. Transformation of mycosis fungoides/Sezary syndrome: clinical characteristics and prognosis. Blood. 1998 Aug. 15; 92(4):1150-9.
   Durante M A, Rodriguez D A, Kurtenbach S, Kuznetsov J N, Sanchez M I, Decatur C L, et al. Single-cell analysis reveals new evolutionary complexity in uveal melanoma. Nat Commun 2020; 11(1):496 doi 10.1038/s41467-019-14256-1.
Ebright R Y, Lee S, Wittner B S, Niederhoffer K L, Nicholson B T, Bardia A, et al. Deregulation of ribosomal protein expression and translation promotes breast cancer metastasis. Science 2020; 367(6485):1468-73 doi 10.1126/science.aay0939.
Efremova M, Vento-Tormo M, Teichmann S A, Vento-Tormo R. CellPhoneDB: inferring cell-cell communication from combined expression of multi-subunit ligand-receptor complexes. Nat Protoc 2020; 15(4):1484-506 doi 10.1038/s41596-020-0292-x.
Ellrott K, Bailey MH, Saksena G, Covington KR, Kandoth C, Stewart C, et al. Scalable Open Science Approach for Mutation Calling of Tumor Exomes Using Multiple Genomic Pipelines. Cell Syst 2018; 6(3):271-81 e7 doi 10.1016/j.cels.2018.03.002.
Gaydosik A M, Tabib T, Geskin L J, Bayan CA, Conway J F, Lafyatis R, Fuschiotti P. Single-Cell Lymphocyte Heterogeneity in Advanced Cutaneous T-cell Lymphoma Skin Tumors. Clin Cancer Res. 2019 Jul. 15; 25(14):4443-4454.
Goetzman E S, Prochownik E V. The Role for Myc in Coordinating Glycolysis, Oxidative Phosphorylation, Glutaminolysis, and Fatty Acid Metabolism in Normal and Neoplastic Tissues. Front Endocrinol (Lausanne) 2018; 9:129 doi 10.3389/fendo.2018.00129.
Greenlee R T, Goodman M T, Lynch C F, Platz C E, Havener L A, Howe H L. The occurrence of rare cancers in U.S. adults, 1995-2004. Public Health Rep 2010; 125(1):28-43 doi 10.1177/003335491012500106.
Hamrouni A, Fogh H, Zak Z, Odum N, Gniadecki R. Clonotypic Diversity of the T-cell Receptor Corroborates the Immature Precursor Origin of Cutaneous T-cell Lymphoma. Clin Cancer Res 2019; 25(10):3104-14 doi 10.1158/1078-0432.CCR-18-4099.
Han H, Jain A D, Truica M I, Izquierdo-Ferrer J, Anker J F, Lysy B, et al. Small-Molecule MYC Inhibitors Suppress Tumor Growth and Enhance Immunotherapy. Cancer Cell 2019; 36(5):483-97 e15 doi 10.1016/j.ccell.2019.10.001.
Herrera A, Cheng A, Mimitou EP, Seffens A, George DD, Bar-Natan M, et al. Multimodal single-cell analysis of cutaneous T cell lymphoma reveals distinct sub-clonal tissue-dependent signatures. Blood 2021 doi 10.1182/blood.2020009346.
Hinds G A, Heald P. Cutaneous T-cell lymphoma in skin of color. J Am Acad Dermatol. 2009 March;60(3):359-75; quiz 376-8.
Iyer A, Hennessey D, O'Keefe S, Patterson J, Wang W, Wong G K, et al. Skin colonization by circulating neoplastic clones in cutaneous T-cell lymphoma. Blood 2019; 134(18):1517-27 doi 10.1182/blood.2019002516.
Jawed S I, Myskowski P L, Horwitz S, Moskowitz A, Querfeld C. Primary cutaneous T-cell lymphoma (mycosis fungoides and Sezary syndrome): part I. Diagnosis: clinical and histopathologic features and new molecular and biologic markers. J Am Acad Dermatol 2014; 70(2):205 el-16; quiz 21-2 doi 10.1016/j.jaad.2013.07.049.
Johnson W T, Kartan S, Nikbakht N, Porcu P. A Prospective Cohort of Black Patients with Mycosis Fungoides and Sézary Syndrome: Subset Analysis of the Mavoric Trial. Blood (2019) 134 (Supplement_1): 4766.
Jones C L, Degasperi A, Grandi V, Amarante T D, Genomics England Research C, Mitchell T J, et al. Spectrum of mutational signatures in T-cell lymphoma reveals a key role for UV radiation in cutaneous T-cell lymphoma. Sci Rep 2021; 11(1):3962 doi 10.1038/s41598-021-83352-4.
Keren L, Bosse M, Thompson S, Risom T, Vijayaragavan K, McCaffrey E, Marquez D, Angoshtari R, Greenwald N F, Fienberg H, Wang J, Kambham N, Kirkwood D, Nolan G, Montine TJ, Galli S J, West R, Bendall S C, Angelo M. MIBI-TOF: A multiplexed imaging platform relates cellular phenotypes and tissue structure. Sci Adv. 2019 Oct. 9; 5(10): eaax5851.
Kiel M J, Sahasrabuddhe A A, Rolland D C, et al. Genomic analyses reveal recurrent mutations in epigenetic modifiers and the JAK-STAT pathway in Sezary syndrome. Nat Commun. 2015; 6:8470.
Kim Y H, Bagot M, Pinter-Brown L, Rook A H, Porcu P, Horwitz S M, Whittaker S, Tokura Y, Vermeer M, Zinzani P L, Sokol L, Morris S, Kim E J, Ortiz-Romero P L, Eradat H, Scarisbrick J, Tsianakas A, Elmets C, Dalle S, Fisher D C, Halwani A, Poligone B, Greer J, Fierro M T, Khot A, Moskowitz A J, Musiek A, Shustov A, Pro B, Geskin L J, Dwyer K, Moriya J, Leoni M, Humphrey J S, Hudgens S, Grebennik D O, Tobinai K, Duvic M; MAVORIC Investigators. Mogamulizumab versus vorinostat in previously treated cutaneous T-cell lymphoma (MAVORIC): an international, open-label, randomised, controlled phase 3 trial. Lancet Oncol. 2018 September; 19(9):1192-1204.
Kim Y H, Khodadoust M S, de Masson A, Moins-Teisserenc H, Ito T, Dwyer K, Herr F, Bagot M. Patient Characteristics of Long-Term Responders to Mogamulizumab: Results from the MAVORIC Study. In: 62^{nd} American Society of Hematology Annual Meeting and Exposition; Dec. 5-8, 2020; San Diego, CA. Abstract #2082.
Kim Y H, Prince H M, Whittaker S, Horwitz S M, Duvic M, Bechter 0, et al. Response to brentuximab vedotin versus physician's choice by CD30 expression and large cell transformation status in patients with mycosis fungoides: An ALCANZA sub-analysis. Eur J Cancer 2021; 148:411-21 doi 10.1016/j.ejca.2021.01.054.
Kucuk C, Jiang B, Hu X, Zhang W, Chan J K, Xiao W, et al. Activating mutations of STAT5B and STAT3 in lymphomas derived from gammadelta-T or NK cells. Nat Commun 2015; 6:6025 doi 10.1038/ncomms7025.
Lawrence M S, Stojanov P, Polak P, Kryukov G V, Cibulskis K, Sivachenko A, et al. Mutational heterogeneity in cancer and the search for new cancer-associated genes. Nature 2013; 499(7457):214-8 doi 10.1038/naturel2213.
Lee S, Zhao L, Rojas C, Bateman N W, Yao H, Lara O D, et al. Molecular Analysis of Clinically Defined Subsets of High-Grade Serous Ovarian Cancer. Cell Rep 2020; 31(2):107502 doi 10.1016/j.celrep.2020.03.066.
Levine J H, Simonds E F, Bendall S C, Davis K L, Amir el A D, Tadmor M D, et al. Data-Driven Phenotypic Dissection of AML Reveals Progenitor-like Cells that Correlate with Prognosis. Cell 2015; 162(1):184-97 doi 10.1016/j.cell.2015.05.047.
Martincorena I, Raine K M, Gerstung M, Dawson K J, Haase K, Van Loo P, et al. Universal Patterns of Selection in Cancer and Somatic Tissues. Cell 2017; 171(5):1029-41 e21 doi 10.1016/j.cell.2017.09.042.
Mayakonda A, Lin D C, Assenov Y, Plass C, Koeffler H P. Maftools: efficient and comprehensive analysis of somatic variants in cancer. Genome Res 2018; 28(11):1747-56 doi 10.1101/gr.239244.118.
McGirt L Y, Jia P, Baerenwald D A, et al. Whole-genome sequencing reveals oncogenic mutations in mycosis fungoides. Blood. 2015; 126(4):508-519.
Mermel C H, Schumacher S E, Hill B, Meyerson M L, Beroukhim R, Getz G. GISTIC2.0 facilitates sensitive and confident localization of the targets of focal somatic copy-number alteration in human cancers. Genome Biol 2011; 12(4):R41 doi 10.1186/gb-2011-12-4-r41.
Miles L A, Bowman R L, Merlinsky T R, Csete I S, Ooi A T, Durruthy-Durruthy R, Bowman M, Famulare C, Patel M A, Mendez P, Ainali C, Demaree B, Delley C L, Abate A R, Manivannan M, Sahu S, Goldberg A D, Bolton K L, Zehir A, Rampal R, Carroll M P, Meyer S E, Viny A D, Levine R L. Single-cell mutation analysis of clonal evolution in myeloid malignancies. Nature. 2020 November; 587(7834):477-482.
Miranda A, Hamilton P T, Zhang A W, Pattnaik S, Becht E, Mezheyeuski A, et al. Cancer stemness, intratumoral heterogeneity, and immune response across cancers. Proc Natl Acad Sci USA 2019; 116(18):9020-9 doi 10.1073/pnas.1818210116.
Mirza A S, Homa P, Teer J K, Song J, Akabari R, Hussaini M, Sokol L. New Insights Into the Complex Mutational Landscape of Sezary Syndrome. Front Oncol. 2020 Apr. 21; 10:514.
Molina J R, Sun Y, Protopopova M, Gera S, Bandi M, Bristow C, et al. An inhibitor of oxidative phosphorylation exploits cancer vulnerability. Nat Med 2018; 24(7):1036-46 doi 10.1038/s41591-018-0052-4.
Monaco G, Lee B, Xu W, Mustafah S, Hwang Y Y, Carre C, et al. RNA-Seq Signatures Normalized by mRNA Abundance Allow Absolute Deconvolution of Human Immune Cell Types. Cell Rep 2019; 26(6):1627-40 e7 doi 10.1016/j.celrep.2019.01.041.
Morita K, Wang F, Jahn K, Hu T, Tanaka T, Sasaki Y, Kuipers J, Loghavi S, Wang S A, Yan Y, Furudate K, Matthews J, Little L, Gumbs C, Zhang J, Song X, Thompson E, Patel K P, Bueso-Ramos C E, DiNardo C D, Ravandi F, Jabbour E, Andreeff M, Cortes J, Bhalla K, Garcia-Manero G, Kantarjian H, Konopleva M, Nakada D, Navin N, Beerenwinkel N, Futreal P A, Takahashi K. Clonal evolution of acute myeloid leukemia revealed by high-throughput single-cell genomics. Nat Commun. 2020 Oct. 21; 11(1):5327.
Mundra P A, Dhomen N, Rodrigues M, Mikkelsen L H, Cassoux N, Brooks K, et al. Ultraviolet radiation drives mutations in a subset of mucosal melanomas. Nat Commun 2021; 12(1):259 doi 10.1038/s41467-020-20432-5.
Nath S K, Yu J B, Wilson L D. Poorer prognosis of African-American patients with mycosis fungoides: an analysis of the SEER dataset, 1988 to 2008. Clin Lymphoma Myeloma Leuk 2014; 14(5):419-23 doi 10.1016/j.clml.2013.12.018.
Neftel C, Laffy J, Filbin M G, Hara T, Shore M E, Rahme G J, et al. An Integrative Model of Cellular States, Plasticity, and Genetics for Glioblastoma. Cell 2019; 178(4):835-49 e21 doi 10.1016/j.cell.2019.06.024.
Olshen A B, Venkatraman E S, Lucito R, Wigler M. Circular binary segmentation for the analysis of array-based DNA copy number data. Biostatistics 2004; 5(4):557-72 doi 10.1093/biostatistics/kxh008.
Park J, Daniels J, Wartewig T, Ringbloom K G, Martinez-Escala M E, Choi S, et al. Integrated Genomic Analyses of Cutaneous T Cell Lymphomas Reveal the Molecular Bases for Disease Heterogeneity. Blood 2021 doi 10.1182/blood.2020009655.
Park J, Yang J, Wenzel A T, Ramachandran A, Lee W J, Daniels J C, Kim J, Martinez-Escala E, Amankulor N, Pro B, Guitart J, Mendillo M L, Savas J N, Boggon T J, Choi J. Blood. 2017 Sep. 21; 130(12):1430-1440.
Pastushenko I, Mauri F, Song Y, de Cock F, Meeusen B, Swedlund B, et al. Fatl deletion promotes hybrid EMT state, tumour stemness and metastasis. Nature 2021; 589(7842):448-55 doi 10.1038/s41586-020-03046-1.
Patient Characteristics of Long-Term Responders to Mogamulizumab: Results from the MAVORIC Study [Poster]. Kim Y H, Khodadoust M S, Masson A, Moins-Teisserenc H, Ito T, Dwyer K, et al.2020.
Pham H T T, Maurer B, Prchal-Murphy M, Grausenburger R, Grundschober E, Javaheri T, et al. STAT5BN642H is a driver mutation for T cell neoplasia. J Clin Invest 2018; 128(1):387-401 doi 10.1172/JC194509.
Philips D, Matusiak M, Gutierrez B, Bhate S S, Barlow G L, Jiang S, Demeter J, Smythe K S, Pierce R H, Fling S P, Ramchurren N, Cheever M A, Goltsev Y, West R B, Khodadoust M S, Kim Y H, Schürch CM, Nolan G P. Immune cell topography predicts response to PD-1 blockade in cutaneous T cell lymphoma.
Prasad A, Rabionet R, Espinet B, et al. Identification of gene mutations and fusion genes in patients with Sézary syndrome. J Invest Dermatol. 2016; 136(7):1490-1499.
Prince H M, Kim Y H, Horwitz S M, Dummer R, Scarisbrick J, Quaglino P, et al. Brentuximab vedotin or physician's choice in CD30-positive cutaneous T-cell lymphoma (ALCANZA): an international, open-label, randomised, phase 3, multicentre trial. Lancet 2017; 390(10094):555-66 doi 10.1016/S0140-6736(17)31266-7.
Pulitzer M, Myskowski P L, Horwitz S M, et al. Mycosis fungoides with large cell transformation: clinicopathological features and prognostic factors. Pathology. 2014; 46(7):610-616.
Qiu X, Mao Q, Tang Y, Wang L, Chawla R, Pliner H A, et al. Reversed graph embedding resolves complex single-cell trajectories. Nat Methods 2017; 14(10):979-82 doi 10.1038/nmeth.4402.
Rosenthal R, McGranahan N, Herrero J, Taylor B S, Swanton C. DeconstructSigs: delineating mutational processes in single tumors distinguishes DNA repair deficiencies and patterns of carcinoma evolution. Genome Biol 2016; 17:31 doi 10.1186/s13059-016-0893-4.
Sakamoto Y, Ishida T, Masaki A, Murase T, Yonekura K, Tashiro Y, et al. CCR4 mutations associated with superior outcome of adult T-cell leukemia/lymphoma under mogamulizumab treatment. Blood 2018; 132(7):758-61 doi 10.1182/blood-2018-02-835991.
Sanchez-Vega F, Mina M, Armenia J, Chatila W K, Luna A, La K C, et al. Oncogenic Signaling Pathways in The Cancer Genome Atlas. Cell 2018; 173(2):321-37 e10 doi 10.1016/j.cell.2018.03.035.
Scarisbrick J J, Prince H M, Vermeer M H, Quaglino P, Horwitz S, Porcu P, et al. Cutaneous Lymphoma International Consortium Study of Outcome in Advanced Stages of Mycosis Fungoides and Sezary Syndrome: Effect of Specific Prognostic Markers on Survival and Development of a Prognostic Model. J Clin Oncol 2015; 33(32):3766-73 doi 10.1200/JCO.2015.61.7142.
Su C, Nguyen K A, Bai H X, Cao Y, Tao Y. Xiao R, Karakousis G, Zhang P J, Zhang G. Racial disparity in mycosis fungoides: An analysis of 4495 cases from the US National Cancer Database. J Am Acad Dermatol. 2017 September; 77(3):497-502.
Talpur R, Sui D, Gangar P, Dabaja B S, Duvic M. Retrospective Analysis of Prognostic Factors in 187 Cases of Transformed Mycosis Fungoides. Clin Lymphoma Myeloma Leuk 2016; 16(1):49-56 doi 10.1016/j.clml.2015.11.010.
Traag V A, Waltman L, van Eck N J. From Louvain to Leiden: guaranteeing well-connected communities. Sci Rep 2019; 9(1):5233 doi 10.1038/s41598-019-41695-z.
Trapnell C, Cacchiarelli D, Grimsby J, Pokharel P, Li S, Morse M, et al. The dynamics and regulators of cell fate decisions are revealed by pseudotemporal ordering of single cells. Nat Biotechnol 2014; 32(4):381-6 doi 10.1038/nbt.2859.
Trucco L D, Mundra P A, Hogan K, Garcia-Martinez P, Viros A, Mandal A K, et al. Ultraviolet radiation-induced DNA damage is prognostic for outcome in melanoma. Nat Med 2019; 25(2):221-4 doi 10.1038/s41591-018-0265-6.
Ungewickell A, Bhaduri A, Rios E, et al. Genomic analysis of mycosis fungoides and Sezary syndrome identifies recurrent alterations in TNFR2. Nat Genet. 2015; 47(9):1056-1060.
van der Windt G J, Pearce E L. Metabolic switching and fuel choice during T-cell differentiation and memory development. Immunol Rev 2012; 249(1):27-42 doi 10.1111/j.1600-065X.2012.01150.x.
Vaque J P, Gomez-Lopez G, Monsalvez V, Varela I, Martinez N, Perez C, et al. PLCG1 mutations in cutaneous T-cell lymphomas. Blood 2014; 123(13):2034-43 doi 10.1182/blood-2013-05-504308.
Wang L, Ni X, Covington K R, et al. Genomic profiling of Sezary syndrome identifies alterations of key T cell signaling and differentiation genes. Nat Genet. 2015; 47(12):1426-1434.
Willemze R, Cerroni L, Kempf W, Berti E, Facchetti F, Swerdlow S H, et al. The 2018 update of the WHO-EORTC classification for primary cutaneous lymphomas. Blood 2019; 133(16):1703-14 doi 10.1182/blood-2018-11-881268.
Woollard W J, Pullabhatla V, Lorenc A, et al. Candidate driver genes involved in genome maintenance and DNA repair in Sezary syndrome. Blood. 2016; 127(26):3387-3397.
Yu G, Wang L G, Han Y, He Q Y. clusterProfiler: an R package for comparing biological themes among gene clusters. OMICS 2012; 16(5):284-7 doi 10.1089/omi.2011.0118.
Yuen G J, Demissie E, Pillai S. B lymphocytes and cancer: a love-hate relationship. Trends Cancer 2016; 2(12):747-57 doi 10.1016/j.trecan.2016.10.010.
Yumeen S, Girardi M. Insights Into the Molecular and Cellular Underpinnings of Cutaneous T Cell Lymphoma. Yale J Biol Med 2020; 93(1):111-21.
Zhang L, Yao Y, Zhang S, Liu Y, Guo H, Ahmed M, et al. Metabolic reprogramming toward oxidative phosphorylation identifies a therapeutic target for mantle cell lymphoma. Sci Transl Med 2019; 11(491) doi 10.1126/scitranslmed.aaul167.
Zhou Y, Yang D, Yang Q, Lv X, Huang W, Zhou Z, et al. Single-cell RNA landscape of intratumoral heterogeneity and immunosuppressive microenvironment in advanced osteosarcoma. Nat Commun 2020; 11(1):6322 doi 10.1038/s41467-020-20059-6.

1. A method of detecting malignant cells in a tumor microenvironment comprising obtaining a tissue sample, performing whole exome sequencing (WES), parallel single-cell RNAseq, single cell TCRseq and/or copy number variation (CNV) interference assay on the sample; wherein the pattern of increase or decrease in gene expression and/or the presence of mutations relative to a control indicates that the subject does or does not have a malignancy.

2. The method of claim 1, wherein the tissue sample comprises T cells or B cells.

3. The method of claim 1, wherein a malignancy is indicated by mutations in genes related to Oxidative phosphorylation, MYC targets V1, MYC targets V2, epithelial mesenchymal transition, E2F targets, xenobiotic metabolism, coagulation, WNTb catenin signaling, cholesterol homeostasis, spermatogenesis, estrogen response late, DNA repair

4. The method of claim 1, wherein the cancer is Cutaneous T-cell lymphoma (CTCL) and the presence of a malignancy is indicated by an increase in 5 or more of the genes selected from the list consisting of CD9, RAB25, LSR, CLDN7, EPCAM, TWIST1, LGALS3, S100A6, SLC25A5, PIM3, IL22, PTHLH, CCR7, AHR, CORO1B, ROMO1, MIF, NME2, NDUFB2, SRM, LAIR2, HTRA1, PAGE5, IGFBP2, AREG, XCL2, and XCL1; and a decrease in the expression of 5 or more of the genes consisting of RGCC, ANXA1, FOS, ZFP36, HLA-DPB1, HLA-DPA1, HLA-DRB1, SAT1, FTH1, HSPA1B, DNAJB1, GLIPR1, IL10RA, SELL, TXNIP, ARID5B, RHOH, BTG1, TPT1, HLA-B, HLA-A, HLA-F, TNFRSF4, IL2RA, and GBP5.

5. The method of claim 1, wherein the cancer malignancy is mycosis fungoides (MF), wherein at least on assay comprises copy number variation (CNV), and the presence of the malignancy is indicated by an increase in the expression of four or more of the genes selected from the group consisting of SYCP1, TM4SF1, AHR, NDUFB2, TWIST1, LGALS3, RBBP6, and NDUFA7 and a decrease of six or more of the genes selected from the group consisting of PRAMEF, PCDHGB, PMS2P1, RECQL4, IFNA, CDKN2, NOTCH1, HRAS, POLE, AKT1, DEFB, and SOCS7.

6. The method of claim 1, wherein the cancer malignancy is mycosis fungoides (MF) and the presence of the malignancy is indicated by a mutation in three of more of the genes selected from the group consisting of FSIP2, TEKT4, PCDH15, TP53, STAT3, JAK3, KMT2C, CREBBP, ARID1A, TRRAP, KMT2D, PLCG1, and CDKN2A.

7. The method of claim 1, wherein the cancer malignancy is leukemic CTLC; wherein at least on assay comprises copy number variation (CNV), and the presence of malignancy is indicated by an increase in the expression of CARD11 and/or MUC16 and a decrease in the expression of 5 or more of the genes selected from the group consisting of ARID1A, TNFAIP3, CDKN2A, DAPL1, WT1, ATM, CDKN1B, SOCS2, RB1, and STK11.

8. The method of claim 1, wherein when a malignant cell is detected in the tumor microenvironment, the method further comprises administering to the subject one or more anti-cancer agents.

9. The method of claim 8, wherein the anti-cancer agent comprises an OXPHOS, Myc, MIF, and/or CDK4/6 inhibitor.

10. The method of claim 9, wherein the CDK4/6 inhibitor comprises Palbociclib.

11. A method of diagnosing a cancer type in a subject comprising obtaining a tissue sample, performing whole exonme sequencing (WES), parallel single-cell RNAseq, single cell TCRseq and/or copy number variation (CNV) interference assay on the sample; wherein the pattern of gene expression indicates the type of cancer the subject has.

12. The method of claim 11, wherein the presence of a Cutaneous T-cell lymphoma (CTCL) is indicated by an increase in 5 or more of the genes selected from the list consisting of CD9, RAB25, LSR, CLDN7, EPCAM, TWIST1, LGALS3, S100A6, SLC25A5, PIM3, IL22, PTHLH, CCR7, AHR, CORO1B, ROMO1, MIF, NME2, NDUFB2, SRM, LAIR2, HTRA1, PAGE5, IGFBP2, AREG, XCL2, and XCL1; and a decrease in the expression of 5 or more of the genes consisting of RGCC, ANXA1, FOS, ZFP36, HLA-DPB1, HLA-DPA1, HLA-DRB1, SAT1, FTH1, HSPA1B, DNAJB1, GLIPR1, IL10RA, SELL, TXNIP, ARID5B, RHOH, BTG1, TPT1, HLA-B, HLA-A, HLA-F, TNFRSF4, IL2RA, and GBP5.

13. The method of claim 11, wherein at least on assay comprises copy number variation (CNV), and the presence of a leukemic CTLC is indicated by an increase in the expression of CARD11 and/or MUC16 and a decrease in the expression of 5 or more of the genes selected from the group consisting of ARID1A, TNFAIP3, CDKN2A, DAPL1, WT1, ATM, CDKN1B, SOCS2, RB1, and STK11.

14. The method of claim 11, wherein at least on assay comprises copy number variation (CNV), and the presence of a mycosis fungoides (MF) is indicated by an increase in the expression of four or more of the genes selected from the group consisting of SYCP1, TM4SF1, AHR, NDUFB2, TWIST1, LGALS3, RBBP6, and NDUFA7 and a decrease of six or more of the genes selected from the group consisting of PRAMEF, PCDHGB, PMS2P1, RECQL4, IFNA, CDKN2, NOTCH1, HRAS, POLE, AKT1, DEFB, and SOCS7.

15. The method of claim 11, wherein the presence of a mycosis fungoides (MF) is indicated by a mutation in three of more of the genes selected from the group consisting of FSIP2, TEKT4, PCDH15, TP53, STAT3, JAK3, KMT2C, CREBBP, ARID1A, TRRAP, KMT2D, PLCG1, and CDKN2A.

16. The method of claim 11, wherein when a malignant cell a diagnosis of cancer is reached or a cancer is detected, the method further comprises administering to the subject one or more anti-cancer agents.

17. The method of claim 16, wherein the anti-cancer agent comprises an OXPHOS, Myc, MIF, and/or CDK4/6 inhibitor.

18. The method of claim 17, wherein the CDK4/6 inhibitor comprises Palbociclib.

19. A method of treating a cancer in a subject comprising obtaining a tissue sample, performing whole exome sequencing (WES), parallel single-cell RNAseq, single cell TCRseq and/or copy number variation (CNV) interference assay on the sample; wherein the pattern of increase or decrease in gene expression and/or the presence of mutations relative to a control indicates that the subject has a cancer, malignancy, and/or the cancer type; and wherein a cancer, malignancy, and/or the type of cancer in the subject is detected, administering to the subject one or more anti-cancer agents.

20. The method of claim 19, wherein the anti-cancer agent comprises an OXPHOS, Myc, MIF, and/or CDK4/6 inhibitor.

21. The method of claim 20, wherein the CDK4/6 inhibitor comprises Palbociclib.

22. A kit for diagnosing a cancer in a subject or detecting a malignancy in a subject comprising primers and/or probes for the detection of mutations or the expression of five or more genes selected from the group consisting of CD9, RAB25, LSR, CLDN7, EPCAM, TWIST1, LGALS3, S100A6, SLC25A5, PIM3, IL22, PTHLH, CCR7, AHR, CORO1B, ROMO1, MIF, NME2, NDUF1B2, SRM, LAIR2, HTRA1, PAGE5, IGFBP2, AREG, XCL2, XCL1, RGCC, ANXA1, FOS, ZFP36, HLA-DPB1, HLA-DPA1, HLA-DRB1, SAT1, FTH1, HSPA1B, DNA131, GLIPR1, I10RA, SELL, TXNIP, ARID5B, RHOH, BTG1, TPT1, ILA-B, HLA-A, HLA-F, TNFRSF4, IL2RA, GBP5, FSIP2, TEKT4, PCDH15, TP53, STAT3, JAK3, KMT2C, CREBBP, ARID1A, TRRAP, KMT2D, PLCG1, CDKN2A, SYCP1, TM4SF1, AHR, NDUFB2, TWIST1, LGALS3, RBBP6, NDUFA7, PRAMEF, PCDHGB, PMS2P1, RECQL4, IFNA, CDKN2, NOTCH1, HRAS, POLE, AKT1, DEFB, SOCS7, CARD11, MUC16, ARID1A, TNFATP3, CDKN2A, DAPL1, WT1, ATM, CDKN1B, SOCS2, RB1, and STK11.
